SEQUENCE LISTING (17 sequences)

SEQ ID NO: 1 - protein, 300 aa, Human
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 364 aa, Human
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 3 - protein, 1306 aa, human
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 4 - protein, 111 aa, murine
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 5 - protein, 123 aa, murine
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAK

SEQ ID NO: 6 - protein, 15 aa, murine
RASKSVSTSGYSYLH

SEQ ID NO: 7 - protein, 7 aa, murine
LASNLES

SEQ ID NO: 8 - protein, 9 aa, murine
QHSRELPFT

SEQ ID NO: 9 - protein, 10 aa, murine
GFDFSRYWMS

SEQ ID NO: 10 - protein, 17 aa, murine
EINPTSSTINFTPSLKD

SEQ ID NO: 11 - protein, 12 aa, murine
GNYYRYGDAMDY

SEQ ID NO: 12 - protein, 7 aa, murine
DIALTQS

SEQ ID NO: 13 - protein, 7 aa, murine
EVKLLES

SEQ ID NO: 14 - DNA, 717 bp, murine
atggagacagacacactcctgttatgggtactgctgctctgggttccaggttccactggtgacattgcgctgacacagtctcctgcttccttagctgtatctctgggacagagggccaccatctcatgcagggccagcaaaagtgtcagtacatctggctatagttatctgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 15 - protein, 238 aa, murine
METDTLLLWVLLLWVPGSTGDIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 1392 bp, murine
atggattttgggctgattttttttattgttgctcttttaaaaggggtccagtgtgaggtgaagcttctcgagtctggaggtggcctggtgcagcctggaggatccctgaaactctcctgtgcagcctcaggattcgatttcagtagatactggatgagttgggtccggcaggctccagggaaagggctagaatggattggagagattaatccaactagcagtacgataaactttacgccatctctaaaggataaagtcttcatctccagagacaacgccaaaaatacgctgtacctgcaaatgagcaaagtgagatctgaggacacagccctttattactgtgcaagagggaactactataggtacggagatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 17 - protein, 463 aa, murine
MDFGLIFFIVALLKGVQCEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTPSLKDKVFISRDNAKNTLYLQMSKVRSEDTALYYCARGNYYRYGDAMDYWGQGTSVTVSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2022/0008570 A1
(43) Pub. Date: Jan. 13, 2022

(54) COMBINATION OF RADIOIMMUNOTHERAPY AND IMMUNE CHECKPOINT THERAPY IN THE TREATMENT OF CANCER

(71) Applicant: Actinium Pharmaceuticals, Inc., New York, NY (US)

(72) Inventor: Dale Ludwig, Rockaway, NJ (US)

(73) Assignee: Actinium Pharmaceuticals, Inc., New York, NY (US)

(21) Appl. No.: 17/293,663

(22) PCT Filed: Dec. 23, 2019
(86) PCT No.: PCT/US19/68283

§ 371 Date: May 13, 2021

Related U.S. Application Data
(60) Provisional application No. 62/783,510, filed on Dec. 21, 2018.

(51) Int. Cl. A61K 51/10; C07K 16/28; C07K 16/32; A61P 35/00; A61K 45/06

(52) U.S. Cl. CPC A61K 51/1069; C07K 16/2803; C07K 16/289; C07K 16/32; A61K 2039/505; C07K 16/2896; A61P 35/00; A61K 45/06; C07K 16/2878

(57) ABSTRACT

Methods for treating a proliferative disease or disorder by administering a radioimmunotherapy to generate an immune response in combination with immune checkpoint therapy to further enhance the immune response.

### RELATED APPLICATIONS

The present application claims priority to U.S. Provisional Patent Application No. 62/783,510 filed Dec. 21, 2018, the entirety of which is incorporated herewith.

### FIELD OF THE INVENTION

The present invention relates to methods for treating a subject having a proliferative disorder by administration of a radioimmunotherapy and an immune checkpoint therapy.

### BACKGROUND OF THE INVENTION

Cancer is a heterogeneous group of malignant diseases responsible for millions of deaths worldwide each year. In 2018, mortality in the United States due to cancer exceeded 600,000 people. Despite decades of effort, most cancers remain incurable, largely due to the progression from a localized disease to a metastatic disease. Moreover, cancer cells have developed means to evade the standard checkpoints of the immune system. For example, cancer cells have been found to evade immunosurveillance through reduced expression of tumor antigens, downregulation of MEW class I and II molecules leading to reduced tumor antigen presentation, secretion of immunosuppressive cytokines such as TGFb, recruitment or induction of immunosuppressive cells such as regulatory T cells (Treg) or myeloid-derived suppressor cells (MDSC), and overexpression of certain ligands [e.g., programmed death ligand-1 (PD-L1)] that inhibit the host's existing antitumor immunity.

Another major mechanism of immune suppression by cancer cells is a process known as “T-cell exhaustion”, which results from chronic exposure to tumor antigens, and is characterized by the upregulation of inhibitory receptors. These inhibitory receptors serve as immune checkpoints in order to prevent uncontrolled immune reactions. Various immune checkpoints acting at different levels of T cell immunity have been described in the literature, including PD-1 (i.e., programmed cell death protein 1) and its ligands PD-L1 and PD-L2, CTLA-4 (i.e., cytotoxic T-lymphocyte associated protein-4), LAG3 (i.e., Lymphocyte-activation gene 3), B and T lymphocyte attenuator, T-cell immunoglobulin, TIM-3 (i.e., mucin domain-containing protein 3), and V-domain immunoglobulin suppressor of T cell activation.

Enhancing the efficacy of the immune system by therapeutic intervention is a particularly exciting development in cancer treatment. As indicated, checkpoint inhibitors such as CTLA-4 and PD-1 prevent autoimmunity and generally protect tissues from immune collateral damage. In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), and CD28, activate and/or promote the expansion of T-cells. Regulation of the immune system by inhibition or overexpression of these proteins is an area of promising current research. Such regulation, however, has not shown great promise in the treatment of tumors with low mutational burden, i.e., immunologically cold tumors.

Recently, it has been observed that localized radiation therapy may stimulate the immune system and thus modulate systemic regression of certain cancers, which is known as the radiation-induced abscopal effect (Grass, et al. Curr Probl Cancer 2016 40:10-24). That is, targeted radiation therapy was found to minimize or eradicate metastases at distant sites. Local radiation therapy damages the DNA within tumor cells, leading to tumor-cell apoptosis. Tumor antigens released from the dying tumor cells, e.g., neo-antigens, may provide antigenic stimulation that induces anti-tumor specific responses at these distal metastases. This hypothesis is supported by evidence from T cell deficient mice, wherein single tumor nodules were irradiated, and distal antigenically related nodules were found to regress (Demaria S, et al. Int J Radiat Oncol Biol Phys. 2004 58(3):862-70).

Targeted radiation therapy is not without significant drawbacks. Non-cancerous tissues in the path of the radiation are damaged, and non-localized cancers such as metastatic and hematological cancers aren't easily targeted. Additionally, while radiation therapy may provide release of neo-antigens useful for antigenic stimulation, cancer cells have developed mechanisms to evade the host immune system. Moreover, in patients demonstrating T-cell exhaustion, newly released neo-antigens may not prime the immune system to mount a response. Thus, what is needed are improved methods to specifically target and kill cancer cells while simultaneously improving the immune response to neo-antigens released from the targeted cancer cells.

### SUMMARY OF THE INVENTION

The present invention provides improved methods for the treatment of a broad range of cancers based on the use of radioimmunotherapy in combination with immune checkpoint therapies. Administration of radioimmunotherapy may generate an immune response that may be further enhanced by subsequent administration of an immune checkpoint therapy. Alternatively, the suppression of an immune response, such as by T-cell exhaustion, may be removed by administration of an immune checkpoint therapy followed by targeting of certain antigens with radioimmunotherapy. These and other combinations of radioimmunotherapy and immune checkpoint therapy are the objects of the present invention.

Accordingly, the present invention relates to methods for treating a subject having a proliferative disorder, wherein the method comprises administering to the subject a therapeutically effective amount of a radioimmunotherapy and a therapeutically effective amount of an immune checkpoint therapy. The radioimmunotherapy and the immune checkpoint therapy may be administered at the same time or sequentially, e.g., the radioimmunotherapy may be administered before and/or after the immune checkpoint therapy or vice versa. Administration of the radioimmunotherapy and/or immune checkpoint therapy may be according to a dosing schedule, such as once every 1 day, 2 days, 3 days, 4 days, 5 days, 6 days, 7 days, 8 days, 9 days, 10 days, 11 days, 12 days, 14 days, 21 days, or 28 days.

According to certain other aspects, the radioimmunotherapy may be administered 1, 2, 3, or even 4 weeks before the immune checkpoint therapy, after which administration of the immune checkpoint therapy and/or radioimmunotherapy may be according to any of the schemes described herein, i.e, the immune checkpoint therapy and the radioimmunotherapy, if continued, may be administered at the same time or sequentially.

According to certain aspects, the immune checkpoint therapy may be administered 1, 2, 3, or even 4 weeks before the radioimmunotherapy, after which administration of the radioimmunotherapy and/or immune checkpoint therapy may be according to any of the schemes described herein, i.e, the radioimmunotherapy and the immune checkpoint therapy, if continued, may be administered at the same time or sequentially.

The radioimmunotherapy may comprise an antibody against CD19, CD20, CD22, CD30, CD33, CD38, CD45, CD123, CD138, CS-1, B-cell maturation antigen (BCMA), MAGEA3, MAGEA3/A6, KRAS, CLL1, MUC-1, HER2, HER3, DR5, IL13Rα2, and EphA2, EpCam, GD2, GPA7, PSCA, EGFR, EGFRvIII, ROR1, GPC3, CEA, Mesothelin, PSMA, or a combination thereof. The radioimmunotherapy may comprise an antibody against a protein product of a gene mutated in acute myeloid leukemia, wherein the gene is NPM1, Flt3, TP53, CEBPA, KIT, N-RAS, MLL, WT1, IDH1/2, TET2, DNMT3A, ASXL1, or a combination thereof. The radioimmunotherapy may comprise an antibody against CD33, CD38, CD45, HER3, DR5, or a combination thereof.

The radioimmunotherapy comprises a radionuclide label, such as ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ⁶⁴Cu, ²¹²Pb, ⁸⁹Zr, ⁶⁸Ga, and ¹⁰³Pd, or a combination thereof.

According to certain aspects, the radioimmunotherapy comprises an anti-CD33 antibody, anti-CD38 antibody, anti-CD45 antibody, anti-HER3 antibody, anti-DR5 antibody, or a combination thereof, labeled with ¹³¹I or ²²⁵Ac or ¹⁷⁷Lu.

According to certain aspects, more than one radioimmunotherapy may be administered to the patient, such as radioimmunotherapy against any of the antibodies listed hereinabove, and a radioimmunotherapy against a different one of the antibodies listed above. According to certain aspects, a first radioimmunotherapy may be against CD33, CD38, CD45, HER3, or DR5, and a second radioimmunotherapy may be against a different one of CD33, CD38, CD45, HER3, or DR5. When more than one radioimmunotherapy is administered to the patient, they may be administered as a combination (i.e., administration of a single solution comprising both radioimmunotherapies, or administered within a single administration session of both radioimmunotherapies separately). Alternatively, one of the two radioimmunotherapies may be administered before the immune checkpoint therapy and a second of the radioimmunotherapies may be administered after the immune checkpoint therapy, such as according to any of the administration schedules indicated above.

The immune checkpoint therapy may comprise an antibody against CTLA-4, PD-1, TIM-3, VISTA, BTLA, LAG-3, TIGIT, CD28, OX40, GITR, CD137, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or a combination thereof.

According to certain aspects, the immune checkpoint therapy may comprise an antibody against PD-1, PD-L1, PD-L2, CTLA-4, or a combination thereof.

According to certain aspects, the proliferative disorder is a cancer or solid tumor. According to certain aspects, the proliferative disorder is a hematological disease or disorder selected from one or more of multiple myeloma, acute myeloid leukemia, myelodysplastic syndrome, acute lymphoblastic leukemia, Hodgkin's disease, non-Hodgkin's lymphoma, and myeloproliferative neoplasm.

The present invention is also related to pharmaceutical compositions for treating a proliferative disease, or a hematological disease or disorder, wherein the compositions comprise a synergistic combination of a radioimmunotherapy and an immune checkpoint therapy, such as described herein above.

According to certain aspects of the compositions, the radioimmunotherapy may comprise an anti-CD33 antibody, such as lintuzumab, or an anti-CD38 antibody, such as daratumumab, or an anti-CD45 antibody, such as BC8, or an anti-HER3 antibody, or an anti-DR5 antibody, any of which may be labeled with any of the radionuclides described herein, such as ¹³¹I or ²²⁵Ac or ¹⁷⁷Lu, and the immune checkpoint therapy may comprise an antibody against PD-1, PD-L1, PD-L2, CTLA-4, or a combination thereof.

According to certain aspects of the compositions, the radioimmunotherapy may be included in a subject effective amount comprising a total protein content of less than 16 mg/kg body weight of the subject, less than 10 mg/kg body weight of the subject, or less than 6 mg/kg body weight of the subject. When the radioimmunotherapy includes the radionuclide ²²⁵Ac, a total radioactivity content may be 0.1 to 10 uCi/kg body weight of the subject, such as 0.2 to 6 uCi/kg body weight of the subject, or 0.4 to 5 uCi/kg body weight of the subject. When the radioimmunotherapy includes the radionuclide ¹³¹I, a total radioactivity content may be about 25 mCi, or 50 mCi, or 75 mCi, or 100 mCi, or 150 mCi, or 200 mCi, or 250 mCi, or 300 mCi, or 350 mCi, or 400 mCi, or 450 mCi, or 500 mCi, such as from 25 mCi to 500 mCi, or 50 mCi to 500 mCi, or 100 mCi to 500 mCi. When the radioimmunotherapy includes the radionuclide ¹⁷⁷Lu, a total radioactivity content may be less than 500 uCi/kg body weight of the subject, such as 5 uCi/kg to 450 uCi/kg body weight of the subject, or 20 to 250 uCi/kg body weight of the subject.

According to certain aspects of the compositions, the immune checkpoint therapy may be included in a subject effective amount comprising a dose of 0.1 mg/kg to 50 mg/kg of the patient's body weight, such as 0.1-5 mg/kg, or 5-30 mg/kg.

The objects of the present invention will be realized and attained by means of the combinations specifically outlined in the appended claims. The foregoing general description and the following detailed description and examples of this invention are provided to illustrate various aspects of the present invention, and by no means are to be viewed as limiting any of the described embodiments.

### BRIEF DESCRIPTION OF THE DRAWINGS

FIG. 1 provides the amino acid sequence of human CD38 as shown in GenBank accession number NP_001766.

FIG. 2 provides the amino acid sequence of human CD33 as shown in GenBank accession number NP_001763.

FIG. 3 provides the amino acid sequence of the RABC isoform of the human CD45 protein.

FIGS. 4-7 depict combination therapies according to various embodiments of the present invention.

FIG. 8 provides the sequence of the complementarity determining regions (CDRs), framework regions, and variable domain sequences of the light chain (VL; SEQ ID NO. 4) and the heavy chain (VH; SEQ ID NO. 5) of the anti-CD45 mAb BC8, wherein the CDRs are in bold and underlined.

FIG. 9 provides amino acid sequences comprising the CDRs and an N-terminal portion of the light chain and the heavy chain of the anti-CD45 mAb BC8 (SEQ ID NOS. 6-13).

FIG. 10 provides the nucleotide (SEQ ID NO: 14) and amino acid (SEQ ID NO: 15) sequence of the light chain of the anti-CD45-immunoglobulin BC8.

FIG. 11 provides the nucleotide (SEQ ID NO: 16) and amino acid (SEQ ID NO: 17) sequence of the heavy chain of the anti-CD45-immunoglobulin BC8.

### BRIEF DESCRIPTION OF THE SEQUENCES

SEQ ID NO:1 is the amino acid sequence of human CD38 as shown in GenBank accession number NP_001766.

SEQ ID NO:2 is the amino acid sequence of human CD33 as shown in GenBank accession number NP_001763.

SEQ ID NO:3 is the amino acid sequence of the RABC isoform of the human CD45 protein.

SEQ ID NO:4 is the amino acid sequence of the variable domain of the light chain of the anti-CD45 murine immunoglobulin BC8.

SEQ ID NO:5 is the amino acid sequence of the variable domain of the heavy chain of the anti-CD45 murine immunoglobulin BC8.

SEQ ID NO:6 is the amino acid sequence of CDR1 of the light chain of the anti-CD45 murine immunoglobulin BC8.

SEQ ID NO:7 is the amino acid sequence of CDR2 of the light chain of the anti-CD45 murine immunoglobulin BC8.

SEQ ID NO:8 is the amino acid sequence of CDR3 of the light chain of the anti-CD45 murine immunoglobulin BC8.

SEQ ID NO:9 is the amino acid sequence of CDR1 of the heavy chain of the anti-CD45 murine immunoglobulin BC8.

SEQ ID NO:10 is the amino acid sequence of CDR2 of the heavy chain of the anti-CD45 murine immunoglobulin BC8.

SEQ ID NO:11 is the amino acid sequence of CDR3 of the heavy chain of the anti-CD45 murine immunoglobulin BC8.

SEQ ID NO:12 is the amino acid sequence of a portion of the anti-CD45 murine immunoglobulin BC8 comprising the N-terminus of the light chain.

SEQ ID NO:13 is the amino acid sequence of a portion of the anti-CD45 murine immunoglobulin BC8 comprising the N-terminus of the heavy chain.

SEQ ID NO:14 is the nucleotide sequence of the light chain of the anti-CD45 murine immunoglobulin BC8.

SEQ ID NO:15 is the amino acid sequence of the light chain of the anti-CD45 murine immunoglobulin BC8.

SEQ ID NO:16 is the nucleotide sequence of the heavy chain of the anti-CD45 murine immunoglobulin BC8.

SEQ ID NO:17 is the amino acid sequence of the heavy chain of the anti-CD45 murine immunoglobulin BC8.

### DETAILED DESCRIPTION OF THE INVENTION

The present invention uses radioimmunotherapy combined with an immune checkpoint therapy to provide more durable cancer therapies that stimulate the immune response through potent tumor cell killing and release of neo-antigens and enhanced immune response to those neo-antigens.

Cancers with low mutational burden typically do not respond well to immune checkpoint therapies such as antibodies against checkpoint inhibitors. Previous studies have shown that external beam radiation can lead to an abscopal effect in which a non-irradiated tumor, e.g. melanoma or colon cancer, responds to therapy, presumably due to the stimulation of an immune response at the site of the irradiated tumor that is capable of recognizing and attacking distal tumor lesions.

Radioimmunotherapy uses radiolabeled antibodies against tumor-specific antigens to deliver cytotoxic radiation to the targeted tumor cells, which kills the tumor cells mainly by eliciting single or double strand breaks in DNA. In doing so, the tumor may release a number of tumor-specific antigens that prime the immune cells against these antigens. Targeted radioimmunotherapy thus has the potential to reach local and distal tumor sites and facilitate antigen presentation by antigen presenting cells (i.e., dendritic cells, macrophages). As such, targeted radioimmunotherapy may be capable of eliciting an abscopal effect.

Since radioimmunotherapy does not require cell proliferation, nor is susceptible to multi-drug resistance mechanisms, many tumor types are sensitive to this form of therapy, including cancers like leukemia and lymphoma that exhibit a relatively low mutational burden and therefore may be less sensitive to immune modulating therapies like antibodies against PD1 (i.e., programmed cell death protein-1) or CD137. For example, relatively few genes are known to be mutated in acute myeloid leukemia, including: NPM1, Flt3, TP53, CEBPA, KIT, N-RAS, MLL, WT1, IDH1/2, TET2, DNMT3A, and ASXL1. The facilitated release and presentation of these mutated tumor antigens following targeted radioimmunotherapy may enable the establishment of a robust immune response to the cancer cells that would be otherwise inadequate using conventional therapy.

Certain other genes are overexpressed and/or selectively expressed on cells of hematological origin, such as CD33, CD38, and CD45. Targeting of these cell types with radioimmunotherapies against CD33, CD38, and/or CD45 may provide therapy for malignant and non-malignant hematologic diseases or disorders.

Other genes, such as HER3, are overexpressed in several types of cancers such as breast, gastrointestinal, and pancreatic cancers. A correlation between the expression of HER2/HER3 and the progression from a non-invasive stage to an invasive stage of these cancers has been shown. Agents that interfere with HER3-mediated signaling, such as anti-HER3 antibodies, may enable the establishment of a robust immune response to the cancer cells that would be otherwise inadequate using conventional therapy.

Apoptosis is essential to the physiological process of removing unnecessary or damaged cells and maintaining the number of normal cells in vivo. The death receptor 5, DR5, is known to induce apoptosis in cells. The regulatory mechanism of apoptosis is often impaired in cancer or immune diseases. Antibodies against DR5 may act in an agonistic manner on cells (cancer cells or immune disease-related cells) which express the receptor in order to kill the cells.

Immune checkpoint therapies such as antibodies against checkpoint inhibitors PD1 or PD-L1 (i.e., programmed death ligand-1), TIM3 (i.e., T-cell immunoglobulin and mucin-domain containing-3), Lag3 (i.e., Lymphocyte-activation gene 3), or TIGIT (i.e., T cell immunoreceptor with Ig and ITIM domains), are known to release regulatory controls on immune cells, particularly T cells, stimulating suppressed or exhausted T cells. However, in the absence of an active immune response to the tumor, immune checkpoint therapy is relatively ineffective. Immune checkpoint therapy is typically only effective in eliciting a durable response in about 20% of patients across a range of responsive tumors. In many patients, the failure to respond is likely due to a weak or inadequate immune response to the tumor.

The present invention uses targeted radioimmunotherapy in synergistic combination with antibodies against immune checkpoint inhibitors and/or with co-stimulatory therapies that may further activate T cells (GITR, OX40, and CD137). This targeted radioimmunotherapy may be effective across all tumor types, and particularly in those with a relatively low mutational burden and would be amenable for the treatment of both liquid and solid tumors.

Thus, the present invention envisions a combination therapy, including a combination of a radioimmunotherapy and an immune checkpoint therapy. A disruptive therapy such as radioimmunotherapy has the potential to effect sufficient tumor cell death and enable presentation of antigens through release or engulfment by phagocytic antigen presenting cells. Combination with the immune checkpoint therapy (inhibitory and/or co-stimulatory) will lead to sustained activation of an anti-tumor immune response to the newly released neo-antigens (i.e., abscopal effect), and/or may be used to activate an exhausted immune system so that an immune response is possible (i.e., de-repression of immune suppression).

### Definitions and Abbreviations

Unless otherwise defined herein, scientific and technical terms used in connection with the present invention shall have the meanings that are commonly understood by those of ordinary skill in the art. Additionally, in this description and in the appended claims, use of the singular includes the plural and plural encompasses the singular, unless specifically stated otherwise. For example, although reference is made herein to “an” antibody, “a” radioimmunotherapy, and “the” immune checkpoint therapy, one or more of any of these components and/or any other components described herein may be used.

The word “comprising” and forms of the word “comprising”, as used in this description and in the claims, does not limit the present invention to exclude any variants or additions. Additionally, although the present invention has been described in terms of “comprising”, the processes, materials, and compositions detailed herein may also be described as consisting essentially of or consisting of. For example, while certain aspects of the invention have been described in terms of a method comprising administering an effective amount of a radioimmunotherapy and an effective amount of an immune checkpoint therapy, a method “consisting essentially of” or “consisting of” administering an effective amount of a radioimmunotherapy and an effective amount of an immune checkpoint therapy is also within the present scope. In this context, “consisting essentially of” means that any additional components will not materially affect the efficacy of the method.

Moreover, other than in the examples, or where otherwise indicated, all numbers expressing, for example, quantities of ingredients used in the specification are to be understood as being modified in all instances by the term “about”. Accordingly, unless indicated to the contrary, the numerical parameters set forth in the following specification are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques. Thus, the term “about” when used before a numerical designation, e.g., temperature, time, amount, and concentration, including a range, indicates approximations which may vary by ±10%, ±5%, or ±1%.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Publication No. WO 2016/187514, incorporated by reference herein.

In addition, in this invention, antibodies or antibody fragments can be formulated using one or more routinely used pharmaceutically acceptable carriers. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule comprising two heavy chains and two light chains and which recognizes an antigen; (b) polyclonal and monoclonal immunoglobulin molecules; (c) monovalent and divalent fragments thereof (e.g., di-Fab), and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring (e.g., IgG-Fc-silent). Furthermore, antibodies include chimeric antibodies, wholly synthetic antibodies, single chain antibodies, and fragments thereof. Antibodies may be human, humanized or nonhuman.

As used herein, “Immunoreactivity” refers to a measure of the ability of an immunoglobulin to recognize and bind to a specific antigen. “Specific binding” or “specifically binds” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

As used herein, an “anti-CD33 antibody” is an antibody, antibody fragment, peptide, Fab fragment, or aptamer that binds to any available epitope of CD33. According to certain aspects, the anti-CD33 targeting agent is a humanized antibody against CD33, such as lintuzumab (HuM195), gemtuzumab, or vadastuximab. According to certain aspects, the anti-CD33 targeting agent binds to the epitope recognized by the monoclonal antibody “lintuzumab” or “HuM195.” HuM195 is known, as are methods of making it.

An “anti-CD38 antibody” is an antibody that binds to any available epitope of CD38. According to certain aspects, the anti-CD38 antibody binds to the epitope recognized by the monoclonal antibody “daratumumab.” Daratumumab is known, as are methods of making it.

An “anti-CD45 antibody” is an antibody that binds to any available epitope of CD45. According to certain aspects, the anti-CD45 antibody binds to the epitope recognized by the monoclonal antibody “BC8.” BC8 is known, as are methods of making it. The BC8 antibody may be a chimeric antibody (BC8c) that includes constant regions of the heavy and/or light chains of a human IgG1-IgG4 molecule, or human Kappa molecule.

An “anti-HER3 antibody” is an antibody that binds to any available epitope of HER3. According to certain aspects, the anti-HER3 antibody binds to an epitope of HER3 recognized by Patritumab, Seribantumab, Lumretuzumab, Elgemtumab, or GSK2849330. According to certain aspects, the anti-HER3 antibody is a bispecific antibody against any available epitope of HER3/HER2 such as MM-111 and MM-141/Istiratumab from Merrimack Pharmaceuticals, MCLA0-128 from Merus NV, and MEHD7945A/Duligotumab from Genetech.

An “anti-DR5 antibody” is an antibody that binds to any available epitope of DR5. According to certain aspects, the anti-CD5 antibody binds to an epitope of DR5 recognized by the antibody tigatuzumab, conatumumab, or drozitumab.

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, the terms “proliferative disorder” and “cancer” may be used interchangeably and may include, without limitation, a solid cancer (e.g., a tumor) and a hematologic malignancy.

A “hematologic disease” or “hematological disorder” may be taken to refer to at least a blood cancer. Such cancers originate in blood-forming tissue, such as the bone marrow or other cells of the immune system. A hematologic disease or disorder includes, without limitation, leukemias (such as acute myeloid leukemia (AML), acute promyelocytic leukemia, acute lymphoblastic leukemia (ALL), acute mixed lineage leukemia, chronic myeloid leukemia, chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

“Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

According to certain aspects, the radioimmunotherapy disclosed herein comprises a radiolabeled antibody against a hematologically expressed antigen, such as CD33, CD38, CD45, DR5, or HER3. The antibodies may be labeled with a radioisotope. As used herein, a “radioisotope” can be an alpha-emitting isotope, a beta-emitting isotope, and/or a gamma-emitting isotope. Examples of radioisotopes include the following: ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ⁶⁴Cu, ²¹²Pb, ⁸⁹Zr, ⁶⁸Ga, and ¹⁰³Pd, or a combination thereof.

Methods for affixing a radioisotope to an antibody (i.e., “labeling” an antibody with a radioisotope) are known and described, such as in International Publication No. WO 2017/155937, incorporated herein in its entirety.

According to certain aspects, the radioimmunotherapy may be an antibody radiolabeled with ¹³¹I (“¹³¹I-labeled”), and the effective amount may be below, for example, 1200 mCi (i.e., where the amount of ¹³¹I administered to the subject delivers a total body radiation dose of below 1200 mCi). According to certain aspects, when the antibody is ¹³¹I-labeled, the effective amount may be below 1000 mCi, below 750 mCi, below 500 mCi, below 250 mCi, below 200 mCi, below 150 mCi, below 100 mCi, below 50 mCi, below 40 mCi, below 30 mCi, below 20 mCi or below 10 mCi. According to certain aspects of this method, the effective amount of ¹³¹I-labeled antibody is from 10 mCi to 200 mCi. Examples of effective amounts include, without limitation, from 50 mCi to 100 mCi, from 50 mCi to 150 mCi, from 50 mCi to 200 mCi, from 60 mCi to 140 mCi, from 70 mCi to 130 mCi, from 80 mCi to 120 mCi, from 90 mCi to 110 mCi, from 100 mCi to 150 mCi, 50 mCi, 60 mCi, 70 mCi, 80 mCi, 90 mCi, 100 mCi, 110 mCi, 120 mCi, 130 mCi, 140 mCi, 150 mCi, or 200 mCi. According to certain aspects of this method, the effective amount of ¹³¹I-labeled antibody is from 200 mCi to 1200 mCi. Examples of effective amounts include, without limitation, from 200 mCi to 300 mCi, from 200 mCi to 400 mCi, from 200 mCi to 500 mCi, from 200 mCi to 600 mCi, from 200 mCi to 700 mCi, from 200 mCi to 800 mCi, from 200 mCi to 900 mCi, from 200 mCi to 1000 mCi, from 200 mCi to 1100 mCi, from 300 mCi to 1200 mCi, from 400 mCi to 1200 mCi, from 500 mCi to 1200 mCi, from 600 mCi to 1200 mCi, from 700 mCi to 1200 mCi, from 800 mCi to 1200 mCi, from 900 mCi to 1200 mCi, from 1000 mCi to 1200 mCi, 50 mCi, 100 mCi, 150 mCi, 200 mCi, 300 mCi, 400 mCi, 500 mCi, 600 mCi, 700 mCi, 800 mCi, 900 mCi, 1000 mCi, or 1100 mCi.

According to certain aspects, the radioimmunotherapy may be an antibody radiolabeled with ²²⁵AC (“²²⁵Ac-labeled”), and the effective amount may be below, for example, 5.0 μCi/kg (i.e., where the amount of ²²⁵AC administered to the subject delivers a radiation dose of below 5.0 μCi per kilogram of subject's body weight). According to certain aspects, when the antibody is ²²⁵Ac-labeled, the effective amount is below 4.5 μCi/kg, 4.0 μCi/kg, 3.5 μCi/kg, 3.0 μCi/kg, 2.5 μCi/kg, 2.0 μCi/kg, 1.5 μCi/kg, 1.0 μCi/kg, 0.9 μCi/kg, 0.8 μCi/kg, 0.7 μCi/kg, 0.6 μCi/kg, 0.5 μCi/kg, 0.4 μCi/kg, 0.3 μCi/kg, 0.2 μCi/kg, 0.1 μCi/kg or 0.05 μCi/kg. According to certain aspects, when the antibody is ²²⁵Ac-labeled, the effective amount is from 0.05 μCi/kg to 0.1 μCi/kg, from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg. According to certain aspects, when the antibody is ²²⁵Ac-labeled, the effective amount is 0.05 μCi/kg, 0.1 μCi/kg, 0.2 μCi/kg, 0.3 μCi/kg, 0.4 μCi/kg, 0.5 μCi/kg, 0.6 μCi/kg, 0.7 μCi/kg, 0.8 μCi/kg, 0.9 μCi/kg, 1.0 μCi/kg, 1.5 μCi/kg, 2.0 μCi/kg, 2.5 μCi/kg, 3.0 μCi/kg, 3.5 μCi/kg, 4.0 μCi/kg or 4.5 μCi/kg.

According to certain aspects, the radioimmunotherapy may be an antibody radiolabeled with ¹⁷⁷Lu (“¹⁷⁷Lu labeled”), and the effective amount of ¹⁷⁷Lu labeled antibody is below, for example, 12 mCi/kg (i.e., where the amount of ¹⁷⁷Lu-labeled antibody administered to the subject delivers a radiation dose of below 12 mCi per kilogram of subject's body weight).

According to certain aspects, when the antibody is ¹⁷⁷Lu-labeled, the effective amount is below 12 mCi/kg, 11 mCi/kg, 10 mCi/kg, 9 mCi/kg, 8 mCi/kg, 7 mCi/kg, 6 mCi/kg, 5 mCi/kg, 4 mCi/kg, 3 mCi/kg, 2 mCi/kg, 1 mCi/kg, or 0.5 mCi/kg. According to certain aspects, when the antibody is ¹⁷⁷Lu-labeled, the effective amount is at least 0.1 mCi/kg, 0.5 mCi/kg, 1 mCi/kg, 2 mCi/kg, 3 mCi/kg, 4 mCi/kg, 5 mCi/kg, 6 mCi/kg, 7 mCi/kg, 8 mCi/kg, or 9 mCi/kg. According to certain aspects, an ¹⁷⁷Lu-labeled antibody may be administered at a dose that includes any combination of upper and lower limits as described herein, such as from at least 0.1 mCi/kg to below 10 mCi/kg, or from at least 5 mCi/kg to below 8 mCi/kg.

According to aspects of the present invention, the effective amount of the ¹⁷⁷Lu labeled antibody may be used for diagnostic purposes or may be used for therapeutic purposes. As such, the effective diagnostic amount of the ¹⁷⁷Lu-labeled antibody may be below 2.4 mCi/kg, 2.2 mCi/kg, 2 mCi/kg, or 1.8 mCi/kg, or 1.6 mCi/kg, or 1.4 mCi/kg, or 1.2 mCi/kg, or 1.0 mCi/kg, or 0.8 mCi/kg, or 0.6 mCi/kg, or 0.4 mCi/kg, or 0.2 mCi/kg, or 0.1 mCi/kg. The effective therapeutic amount of the 177Lu-labeled antibody may be below 12 mCi/kg, or 10 mCi/kg, or 9 mCi/kg, or 8 mCi/kg, or 7 mCi/kg, or 6 mCi/kg, or 5 mCi/kg, or 4 mCi/kg, or 3 mCi/kg.

According to aspects of the present invention, the effective diagnostic amount of the ¹⁷⁷Lu-labeled antibody is from 50 mCi to 200 mCi, such as from 50 mCi to 100 mCi, or 100 mCi to 150 mCi, or 150 mCi to 200 mCi. According to aspects of the present invention, the effective therapeutic amount of the ¹⁷⁷Lu-labeled antibody is from 200 mCi to 1000 mCi, such as from 200 mCi to 600 mCi, or 400 mCi to 600 mCi, or 200 mCi to 300 mCi, or 300 mCi to 400 mCi, or 400 mCi to 500 mCi, or 500 mCi to 600 mCi, or 600 mCi to 700 mCi, 700 mCi to 800 mCi, 800 mCi to 900 mCi, 900 mCi to 1000 mCi.

While specific radionuclide labels have been disclosed herein, any from the list provided above are contemplated for labeling the antibodies of the radioimmunotherapy.

According to certain aspects of the present invention, the majority of the targeting agent (antibody, antibody fragment, etc.) administered to a subject typically consists of non-labeled targeting agent, with the minority being the labeled targeting agent, such as with any of the labels described herein. The ratio of labeled to non-labeled targeting agent can be adjusted using known methods. Thus, accordingly to certain aspects of the present invention, the radioimmunotherapy may be provided in a total protein amount of up to 100 mg, such as up to 60 mg, such as 5 mg to 45 mg, or a total protein amount of between 0.01 mg/kg patient weight to 15.0 mg/kg patient weight, such as between 0.01 mg/kg patient weight to 1.0 mg/kg, or between 0.2 mg/kg patient weight to 0.6 mg/kg patient weight, or 0.3 mg/kg patient weight, or 0.4 mg/kg patient weight, or 0.5 mg/kg patient weight.

According to certain aspects of the present invention, the radiolabeled antibody comprises the labeled fraction and non-labeled fraction in a ration of labeled:non-labeled of from about 0.01:10 to 1:10, such as 0.01:5 to 0.1:5, or 0.01:3 to 0.1:3, or 0.01:1 to 0.1:1 labeled:non-labeled. Moreover, the radiolabeled antibody may be provided as a single dose composition tailored to a specific patient. See for example administration methods disclosed in International Publication No. WO 2016/187514, incorporated by reference herein in its entirety. According to certain aspects, the radiolabeled antibody may be provided in multiple doses, wherein each dose in the regime may comprise a composition tailored to a specific patient.

This inventive combination of a labeled fraction and a non-labeled fraction of the antibody or other biologic delivery vehicle allows the composition to be tailored to a specific patient, wherein each of the radiation dose and the protein dose of the antibody are personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Chemotherapeutic”, in the context of this invention, shall mean a chemical compound which inhibits or kills growing cells, and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alcaloids, in particular vinblastine, vincristine, vindesine, vinflunine, and vinorelbine.

“Therapeutically effective amount” or “effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the antibody that may deplete or cause a reduction in the overall number of cells expressing the antigen to which the antibody is targeted or reacts or may inhibit growth of cells expressing the antigen.

As used herein, “depleting”, with respect to cells expressing CD33, CD38, or CD45 shall mean to lower the population of at least one type of cells that express of overexpress CD33, CD38, or CD45 (e.g., at least one type of the subject's peripheral blood lymphocytes or at least one type of the subject's bone marrow lymphocytes). According to certain aspects of this invention, a subject's lymphocyte decrease is determined by measuring the subject's peripheral blood lymphocyte level. As such, and by way of example, a subject's lymphocyte population is depleted if the population of at least one type of the subject's peripheral blood lymphocytes is lowered, such as by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

The term “immune checkpoint therapy” refers to a molecule capable of modulating the function of an immune checkpoint protein in a positive or negative way (in particular the interaction between an antigen presenting cell (APC) such as a cancer cell and an immune T effector cell). The term “immune checkpoint” refers to a protein directly or indirectly involved in an immune pathway that under normal physiological conditions is crucial for preventing uncontrolled immune reactions and thus for the maintenance of self-tolerance and/or tissue protection. The one or more immune checkpoint therapies described herein may independently act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response. In the context of the present invention, the term encompasses immune checkpoint therapies capable of down-regulating at least partially the function of an inhibitory immune checkpoint (antagonist) and/or immune checkpoint therapies capable of up-regulating at least partially the function of a stimulatory immune checkpoint (agonist).

“Pharmaceutically acceptable salt” refers to acid addition salts of basic compounds, e.g., those compounds including a basic amino group, and to basic salts of acidic compounds, e.g., those compounds including a carboxyl group, and to amphoteric salts of compounds that include both an acidic and a basic moiety, such that these salts are suitable for administration in vivo, preferably to humans. Various organic and inorganic acids may be used for forming acid addition salts. Pharmaceutically acceptable salts are derived from a variety of organic and inorganic counter ions well known in the art. Pharmaceutically acceptable salts include, when the molecule contains a basic functionality, by way of example only, hydrochloride, hydrobromide, tartrate, mesylate, acetate, maleate, oxalate and the like, and when the molecule contains an acidic functionality, by way of example only, sodium, potassium, calcium, magnesium, ammonium, tetraalkylammonium, N-methylmorpholinium, and the like. In one embodiment, the pharmaceutically acceptable salt of ezatiostat is ezatiostat hydrochloride.

“Synergistic combinations,” as used herein, are combinations of monotherapies that may provide a therapeutic effect that is comparable to the effectiveness of a monotherapy, while reducing adverse side effects, e.g. damage to non-targeted tissues, immune status, and other clinical indicia. Alternatively, synergistic combinations may provide for an improved effectiveness, which may be measured by total tumor cell number, length of time to relapse, and other indicia of patient health.

Synergistic combinations of the present invention combine a radioimmunotherapy and an immune checkpoint therapy. Synergistic combinations of the present invention combine more than one radioimmunotherapy and one or more immune checkpoint therapy.

Although methods and materials similar or equivalent to those described herein can be used in the practice or testing described herein, suitable methods and materials are described below.

Radioimmunotherapy—Targets

Radioimmunotherapies of the present invention include antibodies labeled with a radionuclide, wherein the antibodies may be recombinant, monoclonal, chimeric, humanized, human, or a fragment thereof.

According to certain aspects of the present invention, the radioimmunotherapy includes antibodies against any known tumor-specific antigen, or against antigens that may target specific cell types. Exemplary antigens include mesothelin, TSHR, CD19, CD123, CD22, CD33, CD30, CD45, CD171, CD138, CS-1, CLL-1, GD2, GD3, B-cell maturation antigen (BCMA), Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-1 1Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2), ERBB3 (Her3), death receptor 5 (DR5), MUC1, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MART1, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, GPA7, and IGLL1.

According to certain aspects, the radioimmunotherapy includes antibodies against protein products of genes mutated in acute myeloid leukemia, including: NPM1, Flt3, TP53, CEBPA, KIT, N-RAS, MLL, WT1, IDH1/2, TET2, DNMT3A, and ASXL1.

According to certain aspects, the antigens may be selected from tumors having a standard or even high mutational burden, such melanomas, renal cell carcinomas, and lung cancers.

According to certain aspects, the antigens may be selected from tumors that are known to be immunologically cold. That is, the antigens may be selected from tumors having a low mutational burden, such as antigens expressed by tumors of the pancreas, neuroblastomas, and hematological diseases.

According to certain aspects, the antigen may be hematopoietic in origin, such as an antigen present on a hematological cell or tumor cell having a hematopoietic origin.

According to certain aspects, the antigen may be selected from the group comprising CD19, CD20, CD22, CD30, CD33, CD38, CD45, CD123, CD138, CS-1, B-cell maturation antigen (BCMA), MAGEA3, MAGEA3/A6, KRAS, CLL1, MUC-1, HER2, HER3, DR5, IL13Rα2, and EphA2, EpCam, GD2, GPA7, PSCA, EGFR, EGFRvIII, ROR1, GPC3, CEA, Mesothelin, and PSMA.

According to certain aspects, the antigen may be selected from antigens known to be expressed on cells involved in hematological diseases such as, for example, CD33, CD38, or CD45.

According to certain aspects, the antigen may be selected from CD38, CD33, CD45, DR5, or HER3.

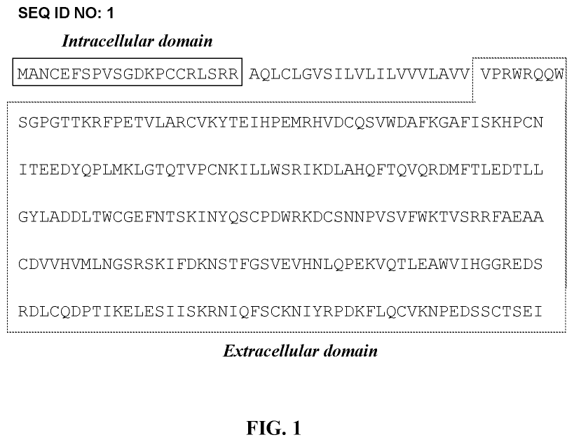

Multiple myeloma cells uniformly overexpress CD38, which is a 45 kD transmembrane glycoprotein. Human CD38 has an amino acid sequence shown in GenBank accession number NP_001766 and in SEQ ID NO: 1 (FIG. 1). As shown in FIG. 1, CD38 is a single pass type II membrane protein with amino acid residues 1-21 representing the cytosolic domain, amino acid residues 22-42 representing the transmembrane domain, and residues 43-300 representing the extracellular domain of CD38. The CD38 protein is a bi-functional ectoenzyme that can catalyze the conversion of NAD⁺ into cyclic ADP-ribose (cADPR), and cADPR into ADP-ribose, and thus regulates extracellular NAD⁺ concentrations. CD38 expression is also upregulated in a variety of malignant hematological diseases, including but not limited to B-cell chronic lymphocytic leukemia, B-cell acute lymphocytic leukemia, Waldenström macroglobulinemia, primary systemic amyloidosis, mantle-cell lymphoma, pro-lymphocytic/myelocytic leukemia, acute myeloid leukemia, chronic myeloid leukemia, follicular lymphoma, NK-cell leukemia and plasma-cell leukemia.

Moreover, expression of CD38 has been described on epithelial/endothelial cells of different origin, including glandular epithelium in prostate, islet cells in pancreas, ductal epithelium in glands, including parotid gland, bronchial epithelial cells, cells in testis and ovary, and tumor epithelium in colorectal adenocarcinoma. Thus, diseases where CD38 expression may also be involved include, but are not limited to, broncho-epithelial carcinomas of the lung, breast cancer evolving from malignant proliferation of the epithelial lining in ducts and lobules of the breast, pancreatic tumors evolving from the b-cells (e.g., insulinomas), and tumors evolving from epithelium in the gut (e.g. adenocarcinoma and squamous cell carcinoma).

According to certain aspects of the present invention, the radioimmunotherapy may comprise a monoclonal antibody against CD38. Exemplary monoclonal antibodies include daratumumab, MOR202, or SAR650984, each of which has been found to bind to a different portion of the extracellular region of CD38, and demonstrate different clinical responses (e.g., anti-tumor effects). Daratumumab, MOR202, or SAR650984 are available from Johnson & Johnson (Janssen Biotech)/Genmab as Darzalex®, Celgene Corp./Morphosys, or Sanofi/Immunogen as Isatuximab, respectively.

Leukemia stem cells, which have been particularly well characterized for acute myeloid leukemia (AML), express a characteristic set of cell-surface antigens including, among others, CD33. The CD33 antigen is expressed on the blast cells of most cases of AML; about 85-90% of AML cases express the CD33 antigen. Moreover, the CD33 antigen is expressed on virtually all cases of chronic myeloid leukemia (CML). Patients older than 60 years have a poor prognosis with only 10% to 15% showing a 4-year disease-free survival for AML. This high relapse rate for AML patients and the poor prognosis for older patients highlight the urgent need for novel therapeutics preferentially targeting CD33⁺ cells.

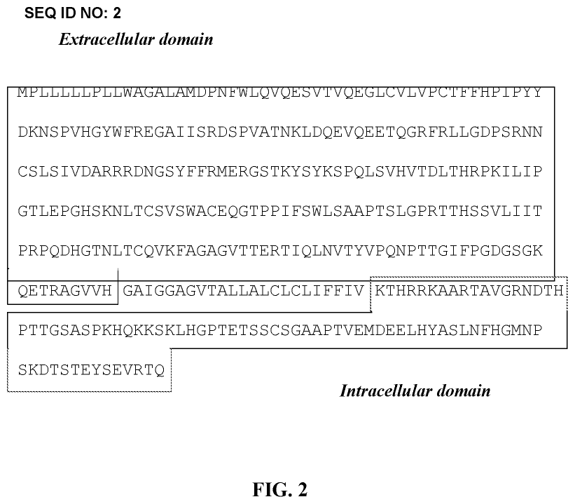

Human CD33 has an amino acid sequence shown in GenBank accession number NP_001763 and in SEQ ID NO: 2 (FIG. 2). CD33 is a 67 Kd type I transmembrane receptor glycoprotein that may function as a sialic acid-dependent cell adhesion molecule. CD33 has a long N-terminal extracellular domain, a helical transmembrane domain, and a short C-terminal cytoplasmic domain. Expressed on early myeloid progenitor and myeloid leukemic (e.g., acute myelogenous leukemia, AML) cells, CD33 is not expressed on stem cells.

With reference to FIG. 2, amino acid residues 1-259 represent the extracellular domain, amino acids 260-282 represent the helical transmembrane domain, and amino acids 283-364 represent the cytosolic domain (intracellular). There are at least three known single nucleotide polymorphisms (“SNPs”) in the extracellular domain of CD33 (i.e., W22R, R69G, S128N). Therefore, the extracellular domain of Homo sapiens CD33 can have the amino acid sequence of SEQ ID NO:2 with any one or more of these SNPs.

Recent studies suggest a role for CD33 in the modulation of inflammatory and immune responses through a dampening effect on tyrosine kinase-driven signaling pathways. For example, in vitro studies have demonstrated that CD33 constitutively suppresses the production of pro-inflammatory cytokines such as IL-1β, TNF-α, and IL-8 by human monocytes in a sialic acid ligand-dependent and SOCS3-dependent manner. Conversely, reduction of cell surface CD33 or interruption of sialic acid binding can increase p38 mitogen-activated protein kinase (MAPK) activity and enhance cytokine secretion as well as cytokine-induced cellular proliferation.

According to certain aspects of the present invention, the radioimmunotherapy may comprise a monoclonal antibody against CD33. Exemplary monoclonal antibodies include lintuzumab (HuM195), gemtuzumab, and vadastuximab, each of which has been found to bind to a different portion of the extracellular region of CD33. Moreover, each of these antibodies demonstrate different clinical responses (e.g., anti-tumor effects). Gemtuzumab is available from Pfizer as Mylotarg™, and vadastuximab is available from Seattle Genetics as Vadastuximab talirine.

For example, the antibody lintuzumab (HuM195) has demonstrated anti-leukemic effects in the treatment of AML. HuM195 is a recombinant humanized anti-CD33 monoclonal antibody originally produced by Protein Design Labs, Inc. (Fremont, Calif.). M195 is a monoclonal IgG2a antibody that binds CD33. M195 is derived from a mouse immunized with live human leukemic myeloblasts. HuM195 was constructed by grafting complementarity-determining regions of M195 into a human IgG1 framework and backbone. HuM195 induced antibody-dependent cell-mediated cytotoxicity using human peripheral blood mononuclear cells as effectors. Four clinical trials have investigated native (i.e., unconjugated) HuM195 alone in patients with relapsed or refractory AML and CML. Fever, chills, and nausea were the most common toxicities. Human anti-human antibody responses were not seen. Beneficial biologic activity in terms of reduction in marrow blast cells was seen in some patients. Those who benefited the most had fewer blasts at the beginning of therapy, suggesting that HuM195 may be more effective in the treatment of minimal residual or cytoreduced disease.

The majority of malignancies of hematologic origin, whether myeloid or lymphoid-derived, express CD45 on the surface of tumor cells to varying degrees. This includes leukemias (such as acute myeloid leukemia (AML), acute promyelocytic leukemia, acute lymphoblastic leukemia (ALL), acute mixed lineage leukemia, chronic myeloid leukemia, chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies.

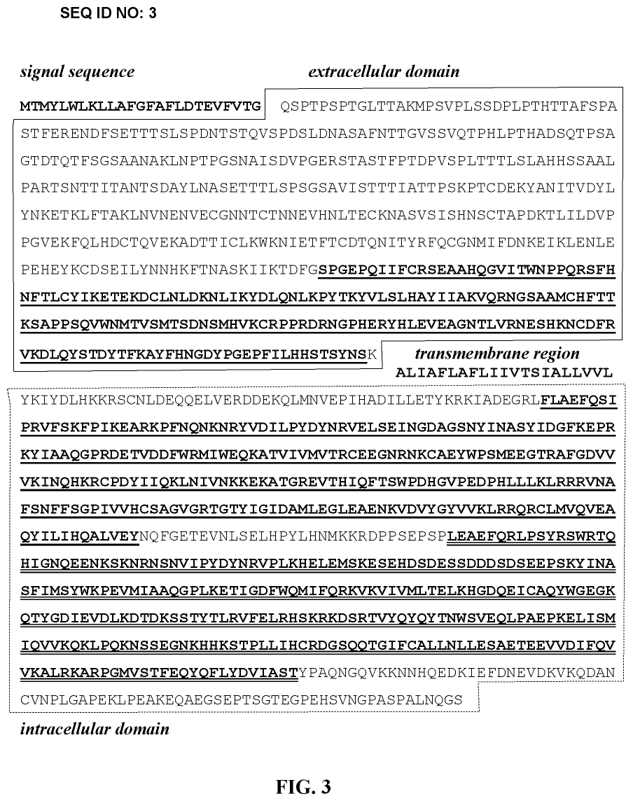

CD45 is not found on tissues of non-hematopoietic origin, making it a good target for the treatment of these malignancies. Among several clones of the anti-CD45 murine antibody, BC8 recognizes all the human isoforms of the CD45 antigen (CD45 RABC isoform shown in FIG. 3), and thus provides an excellent target for the development of therapeutics for human malignancies of hematopoietic origin, including leukemias and lymphomas. Thus, the radioimmunotherapy of the presently disclosed invention may comprise a monoclonal antibody against CD45.

According to certain aspects, the anti-CD45 antibody may comprise the BC8 monoclonal antibody, such as substantially detailed in U.S. Pat. No. 10,420,851, incorporated by reference herein. An exemplary composition comprising the BC8 monoclonal antibody includes those compositions as detailed in WO 2017/155937.

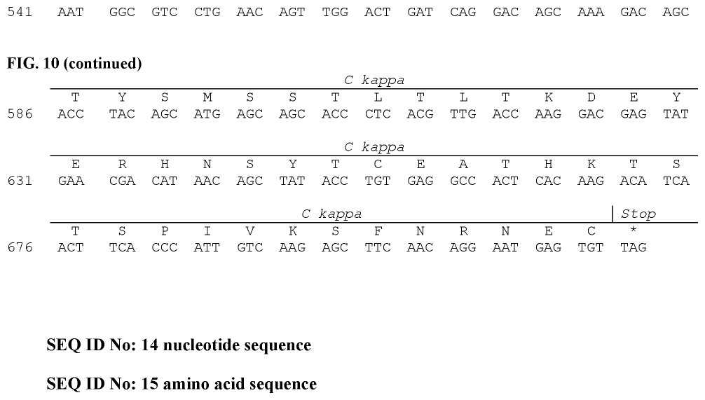

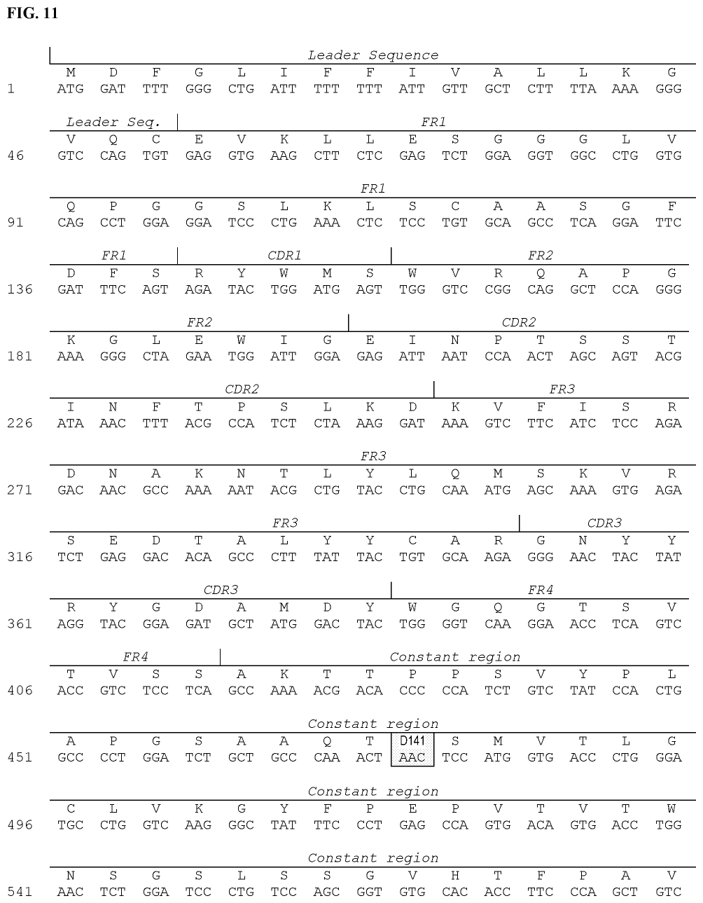

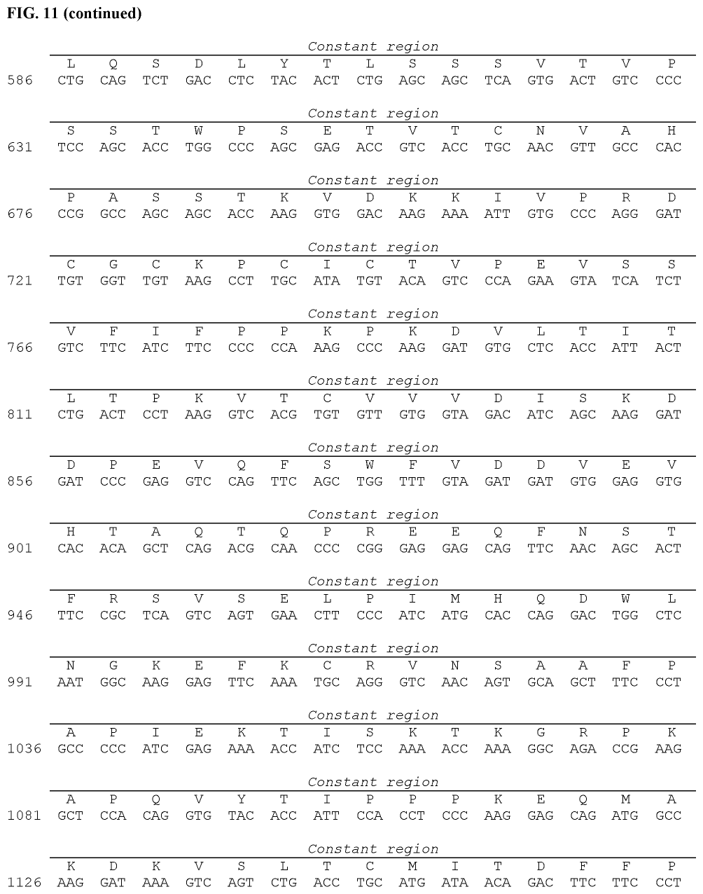

The BC8 monoclonal antibody may have a light chain comprising the amino acid sequence set forth in SEQ ID NO:15 (FIG. 10). The BC8 monoclonal antibody may have a light chain variable region comprising the amino acid sequence set forth in SEQ ID NO:4 (FIG. 8). The BC8 monoclonal antibody may have a light chain comprising the N-terminal amino acid sequence set forth in SEQ ID NO:12 (FIG. 9). According to certain aspects, the light chain includes at least one complementarity determining region comprising the amino acid sequence as set forth in SEQ ID NO:6, SEQ ID NO:7, or SEQ ID NO:8 (FIG. 9). According to certain aspects, the light chain comprises the N-terminal amino acid sequence set forth in SEQ ID NO:12 and at least one complementarity determining region comprising the amino acid sequence as set forth in SEQ ID NO:6, SEQ ID NO:7, or SEQ ID NO:8 (FIG. 9).

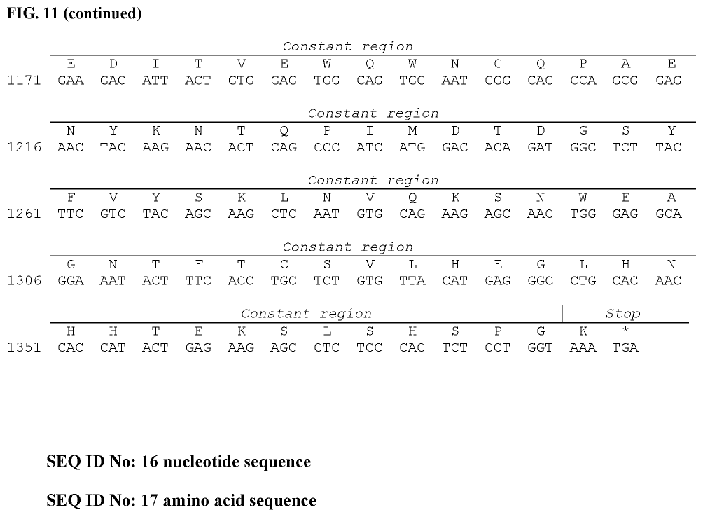

The BC8 monoclonal antibody may have a heavy chain comprising the amino acid sequence set forth in SEQ ID NO:17 (FIG. 11). The BC8 monoclonal antibody may have a heavy chain variable region comprising the amino acid sequence set forth in SEQ ID NO:5 (FIG. 8). The BC8 monoclonal antibody may have a heavy chain comprising the N-terminal amino acid sequence set forth in SEQ ID NO:13 (FIG. 9). According to certain aspects, the heavy chain includes at least one complementarity determining region comprising the amino acid sequence as set forth in SEQ ID NO:9, SEQ ID NO:10, or SEQ ID NO:11 (FIG. 9). According to certain aspects, the heavy chain comprises the N-terminal amino acid sequence set forth in SEQ ID NO:13 and at least one complementarity determining region comprising the amino acid sequence as set forth in SEQ ID NO:9, SEQ ID NO:10, or SEQ ID NO:11 (FIG. 9).

As shown in FIG. 11, the amino acid at position 141 (relative to the N-terminal amino acid) of the BC8 monoclonal antibody heavy chain may be either an ASP or an ASN. As such, a population of BC8 antibody molecules may include both ASP and ASN at position 141.

As shown in FIGS. 10 and 11, the light and heavy chains, respectively, include a leader sequence, and constant regions derived from the specific mouse hybridoma. Any one or more of these regions may be substituted with comparable regions from human antibodies, i.e., human leader sequence, human IgG1-4 constant regions, etc.

Proposed methods by which these antibodies eliminate antigen-positive cells, such as CD45-, CD38-, or CD33-positive cells, include antibody-dependent cellular cytotoxicity (ADCC), complement-dependent cytotoxicity (CDC), and apoptosis.

According to certain aspects, the radioimmunotherapy may be directly involved in apoptotic pathways, such as an agonist of DR5 (death receptor 5; also known as TRAIL-R2, tumor necrosis factor-related apoptosis-inducing ligand-receptor 2). DR5 is known to trigger apoptosis when activated by its ligand, TRAIL (tumor necrosis factor-related apoptosis-inducing ligand). While DR5 is found to be overexpressed in endothelial cells within solid tumors but not normal tissues, the tumor cells are often found to be resistance to TRAIL-induced apoptosis. Activation of DR5 by antibodies against the receptor has been noted to lead to tumor biased cell death. Thus, DR5 presents an excellent target for radioimmunotherapy, both to specifically target the tumor cells and to induce apoptosis of these cells. Exemplary antibodies against DR5 include at least tigatuzumab (CD-1008) from Daiichi Sankyo, conatumumab (AMG 655) from Amgen, and drozitumab from Genentech. Initial studies in mouse models may use the surrogate mouse antibody TRA-8.

According to certain aspects, the radioimmunotherapy may comprise an antibody against human epidermal growth factor receptor 3 (HER3). HER3 is a type I transmembrane glycoprotein that is a member of the erythroblastic oncogene B (ErbB) family of tyrosine kinase receptors (EGFR, HER2, HER3, and HER4). Signaling through HER3 can be activated in a ligand-dependent or ligand-independent manner. In the absence of ligand, HER3 receptor molecules are normally expressed at the cell surface as monomers with a conformation which prevents receptor dimerization in which the dimerization loop of subdomain II makes intramolecular contact with a pocket on subdomain IV. Binding of a HER3 ligand such as a neuregulin (NRG), e.g. NRG1 (also known as heregulin, HRG) or NRG2 to subdomains I and III of the extracellular region causes a conformational change which results in the exposure of the dimerization loop of subdomain II, facilitating receptor dimerization and signaling.

Some cancer-associated mutations in HER3 may disrupt interaction of subdomains II and IV required for the formation of the inactive ‘closed’ conformation and thereby cause constitutive presentation of the dimerization loop and activation of HER3-mediated signaling in the absence of ligand binding. Antibodies that target HER3 may be useful in targeting specific cancer cells, particularly certain solid cancers. Exemplary antibodies against HER3 include monoclonal antibodies such as Patritumab from Daiichi Sankyo, Seribantumab (MM-121) from Merrimack Pharmaceuticals, Lumretuzumab from Roche, Elgemtumab from Novartis, and GSK2849330 from GlaxoSmithKline, and bispecific antibodies against HER3/HER2 such as MM-111 and MM-141/Istiratumab from Merrimack Pharmaceuticals, MCLA0-128 from Merus NV, and MEHD7945A/Duligotumab from Genetech.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer (NK) cells, monocytes, macrophages and neutrophils via Fc gamma receptors (FcγR) expressed on effector cells. For example, NK cells express FcγRIIIa, whereas monocytes express FcγRI, FcγRII and FcvRIIIa. Death of the antibody-coated target cell, such as the CD33-expressing cells, occurs as a result of effector cell activity through the secretion of membrane pore-forming proteins and proteases.

“Complement-dependent cytotoxicity”, or “CDC”, refers to a mechanism for inducing cell death in which an Fc effector domain of a target-bound antibody binds and activates complement component C1q, which in turn activates the complement cascade leading to target cell death. Activation of complement may also result in deposition of complement components on the target cell surface that facilitate ADCC by binding complement receptors (e.g., CR3) on leukocytes.

“Apoptosis” refers to a mechanism of programmed cell death wherein antibody binding to the target cell disrupts integral cell signaling pathways and results in cell self-destruction.

To assess ADCC activity of an antibody that binds to a specific antigen, the antibody may be added to antigen-expressing cells in combination with immune effector cells, which may be activated by the antigen-antibody complexes resulting in cytolysis of the antigen-expressing cells, respectively. Cytolysis is generally detected by the release of a label (e.g. radioactive substrates, fluorescent dyes or natural intracellular proteins) from the lysed cells. Exemplary effector cells for such assays include peripheral blood mononuclear cells (PBMC) and NK cells.

As example, in an exemplary assay for ADCC activity of an anti-CD33 antibody, CD33-expressing cells may be labeled with ⁵¹Cr and washed extensively. The anti-CD33 antibodies may be added to the CD33-expressing cells at various concentrations, and the assay started by adding effector cells (NK cells from peripheral blood mononuclear cells, for example). After incubation for various time intervals at 37° C., assays are stopped by centrifugation and ⁵¹Cr release from lysed cells is measured in a scintillation counter. The percentage of cellular cytotoxicity may be calculated as the percent maximal lysis which may be induced by adding 3% perchloric acid to the CD33-expressing cells.

In an exemplary assay for cytotoxicity, tetrazolium salt may be added to the CD33-expressing cells treated with various amounts of the anti-CD33 antibody. In living mitochondria, the XTT is reduced to an orange product by mitochondrial dehydrogenase and transferred to the cell surface. The orange product can be optically quantified and reflects the number of living cells. Alternatively, esterases from living cells are known to hydrolyze the colorless calcenin into as fluorescent molecule. The fluorescence can be measured and quantified and reflects the number of living cells in the sample. The total amount of dead cells may be measured using propidium iodide, which is excluded from live cells by intact membranes. The fluorescence due to the propidium iodide in dead cells may be quantified by flow-cytometry.

In order to assess CDC, complement protein may need to be included in an assay for cytotoxicity. Measurement of apoptosis induction does not require addition of NK cells or complement protein in an assay for cytotoxicity.

The radioimmunotherapy may include antibodies that are multi-specific. For example, the radioimmunotherapy may include bispecific antibodies against any two different tumor-specific antigens, or two different epitopes of the same antigen. As example, the radioimmunotherapy may comprise a multi-specific antibody against a first epitope of CD33 and a second epitope of CD33, or against an epitope of CD33 and epitopes of one or more additional different antigens, such as an antigen selected from the lists presented above (e.g., CD38, CD45, etc.). As another example, the radioimmunotherapy may include a bispecific antibody against HER3/HER2.

According to certain aspects of the present invention, the radioimmunotherapy includes a multi-specific antibody comprising at least a first target recognition component that specifically binds to an epitope of a first antigen, and a second target recognition component that specifically binds to a different epitope of the first antigen or to an epitope of a second antigen. The multi-specific antibody may be a recombinant antibody, a monoclonal antibody, a chimeric antibody, a humanized antibody, a human antibody, or an antibody fragment.

The first target recognition component may comprise one of: a first full length heavy chain and a first full length light chain, a first Fab fragment, or a first single-chain variable fragment (scFvs). For example, when the first target recognition component is directed to CD33, the first target recognition component may be derived from lintuzumab (HuM195), gemtuzumab, or vadastuximab. The second target recognition component may comprise one of: a second full length heavy chain and a second full length light chain, a second Fab fragment, or a second single-chain variable fragment (scFvs). Moreover, the second target recognition component may be derived from any of the additional different antigens listed above, or from a different epitope of the first target recognition component (i.e., same antigen, different epitope).

Alternatively, the present invention contemplates methods which include administration of more than one radioimmunotherapy. For example, the radioimmunotherapy may comprise a first antibody and at least a second antibody, wherein the first and second antibodies recognize different epitopes of the same antigen or different antigens. For example, the radioimmunotherapy may comprise a first antibody against at least one epitope of CD33, and a second antibody against a different epitope of CD33 than the first antibody, or against an epitope of a different antigen, such as an antigen selected from the lists presented above.

While reference is made herein to CD33 in an exemplary manner, such reference should be understood to include reference to any of the targets of the radioimmunotherapy disclosed herein.

Radioimmunotherapy—Labelling

The radioimmunotherapy of the present invention includes antibodies labeled with a radionuclide so that on treatment of a patient with the radioimmunotherapy, the radionuclide becomes localized to cells expressing the antigen, and induces damage to, and potentially kills, those cells. In addition to the many mechanisms by which antibodies may kill cells, emission of ionizing radiation from a radionuclide labeled antibody may kill cells in close proximity to the antibody bound antigen expressing cells. The radionuclide emits radioactive particles which can damage cellular DNA to the point where the cellular repair mechanisms are unable to allow the cell to continue living. Thus, if the antigen expressing cells are involved in a tumor, the radionuclide may beneficially kill the tumor cells.

Radionuclides that can be used to induce such damage to cells, such as cancer cells, are generally high energy emitters. The high energy radionuclide preferably acts over a short range so that the cytotoxic effects are localized to the targeted cells. In this way, radiotherapy is delivered in a more localized fashion to decrease damage to non-targeted or non-cancerous cells.

Radionuclides useful for labeling the antibodies for use in the radioimmunotherapies of the present invention include ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ⁶⁴Cu, ²¹²Pb, ⁸⁹Zr, ⁶⁸Ga, and ¹⁰³Pd, or a combination thereof.

The antibodies of the present invention may be labeled with the radionuclide by any means known in the art. According to one aspect of the invention, the radionuclide may be attached or chelated by a chelating agent which is conjugated to the antibody such as substantially described in WO 2019/027973, incorporated herein by reference in its entirety. That is, the radionuclide labeled antibody may be prepared by first forming a chelator conjugated antibody (“conjugated antibody”), and then chelating a radionuclide with the conjugated antibody to form the radiolabeled antibody. A radionuclide may be chelated by the conjugated antibody at any time following conjugation.

The chelators useful in the present invention are compounds which have the dual functionality of sequestering metal ions plus the ability to covalently bind a biological carrier such as an antibody. Numerous chelators are known in the art. Exemplary chelators suitable for use in the present invention include, but are not limited to, chelators such as S-2-(4-Isothiocyanatobenzyl)-1,4,7,10 tetraazacyclododecanetetraacetic acid (p-SCN-Bn-DOTA), diethylene triamine pentaacetic acid (DTPA); ethylene diamine tetraacetic acid (EDTA); 1,4,7,10-tetraazacyclododecane-N,N′,N″,N′″-tetraacetic acid (DOTA); p-isothiocyanatobenzyl-1,4,7,10-tetraazacyclododecane-1,4,7,10-te-traacetic acid (p-SCN-Bz-DOTA); 1,4,7,10-tetraazacyclododecane-N,N′,N″-triacetic acid (DO3 A); 1,4,7,10-tetraazacyclododecane-1,4,7,10-tetrakis(2-propionic acid) (DOTMA); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (NOTA); 1,4,8,11-tetraazacyclotetradecane-N,N′,N″,N′″-tetraacetic acid (TETA); triethylene tetraamine hexaacetic acid (TTHA); trans-1,2-diaminohexane tetraacetic acid (CYDTA); 1,4,7,10-tetraazacyclododecane-1-(2-hydroxypropyl)-4,7,10-triacetic acid (HP-DO3A); trans-cyclohexane-diamine tetraacetic acid (CDTA); trans(1,2)-cyclohexane diethylene triamine pentaacetic acid (CDTPA); 1-oxa-4,7,10-triazacyclododecane-N,N′,N″-triacetic acid (OTTA); 1,4,7,10-tetra-azacyclododecane-1,4,7,10-tetrakis {3-(4-carboxyl)-butanoic acid}; 1,4,7,10-tetra-azacyclododecane-1,4,7,10-tetrakis (acetic acid-methyl amide); 1,4,7,10-tetra-azacyclododecane-1,4,7,10-tetrakis (methylene phosphonic acid); and derivatives thereof.

According to certain aspects of the present invention, the radiolabeled antibody may be stable for a time period long enough to produce and administer to a patient (e.g., several days or weeks), but the radionuclide may decay the antibody after it has reached the target cells (cells expressing the antigen) and before it can exert damage to normal cells. For example, it has been found that greater than 75% of a ²²⁵Ac labeled monoclonal antibody against CD33 may remain intact after storage for 24 hours at 4° C. This provides enough time to produce, transport, and administer the radioimmunotherapy, and enough time for the radionuclide to damage the target cells. The ²²⁵Ac labeled anti-CD33 is then decayed before it significantly damages cells not expressing the CD33 antigen.

According to certain aspects of the present invention, the radiolabeled antibody may be prepared as a composition by the methods disclosed in the International Patent Application Publication No. WO2016/187514. Moreover, the radiolabeled antibody may be administered by methods disclosed in the same publication.

According to certain aspects of the present invention, the antibody may be labeled with ²²⁵Ac, ¹³¹I, or ¹⁷⁷Lu, and may be at least 5-fold, 10-fold, 20-fold, 50-fold, or even 100-fold more effective at causing cell death of lymphoblast, myeloma cells, myeloblast cells or malignant plasmacytes, than a control antibody, wherein the control antibody comprises an un-labeled antibody against the same epitope or antigen as the ²²⁵Ac, ¹³¹I, or ¹⁷⁷Lu labeled antibody.

Immune Checkpoint Therapy

Immune checkpoint therapies of the present invention include molecules that totally or partially reduce, inhibit, interfere with or modulate one or more checkpoint proteins. Checkpoint proteins regulate T-cell activation or function. Immune checkpoint therapies may unblock an existing immune response inhibition by binding to or otherwise disabling checkpoint inhibition. The immune checkpoint therapies may include monoclonal antibodies, humanized antibodies, fully human antibodies, antibody fragments, small molecule therapeutics, or a combination thereof.

Exemplary immune checkpoint therapies may specifically bind to and inhibit a checkpoint protein, such as the inhibitory receptors CTLA-4, PD-1, TIM-3, VISTA, BTLA, LAG-3 and TIGIT, and/or the activating receptors CD28, OX40, GITR, CD137, CD27, and HVEM. Additionally, the immune checkpoint therapy may bind to a ligand of any of the aforementioned checkpoint proteins, such as PD-L1, PD-L2, PD-L3, and PD-L4 (ligands for PD-1); CD80 and CD86 (ligands for CTLA-4); CD137-L (ligand of CD137); and GITR-L (ligand of GITR). Other exemplary immune checkpoint therapies may bind to checkpoint proteins such as CD226, B7-H3, B7-H4, BTLA, TIGIT, GALS, KIR, 2B4 (belongs to the CD2 family of molecules and is expressed on all NK, γδ, and memory CD8+ (αβ) T cells), CD160 (also referred to as BY55), and CGEN-15049.

Central to the immune checkpoint process are the CTLA-4 and PD-1 immune checkpoint pathways. The CTLA-4 and PD-1 pathways are thought to operate at different stages of an immune response. CTLA-4 is considered the “leader” of the immune checkpoint inhibitors, as it stops potentially autoreactive T cells at the initial stage of naive T-cell activation, typically in lymph nodes. The PD-1 pathway regulates previously activated T cells at the later stages of an immune response, primarily in peripheral tissues. Moreover, progressing cancer patients have been shown to lack upregulation of PD-L1 by either tumor cells or tumor-infiltrating immune cells. Immune checkpoint therapies targeting the PD-1 pathway might thus be especially effective in tumors where this immune suppressive axis is operational and reversing the balance towards an immune protective environment would rekindle and strengthen a pre-existing anti-tumor immune response. PD-1 blockade can be accomplished by a variety of mechanisms including antibodies that bind PD-1 or its ligand, PD-L1.

For example, the immune checkpoint therapy may comprise an inhibitor of the PD-1 checkpoint, which may decrease, block, inhibit, abrogate or interfere with signal transduction resulting from the interaction of PD-1 with one or more of its binding partners, such as PD-L1 and PD-L2. Moreover, the inhibitor of the PD-1 checkpoint may be an anti-PD-1 antibody, antigen binding fragment, fusion proteins, oligopeptides, and other molecules that decrease, block, inhibit, abrogate or interfere with signal transduction resulting from the interaction of PD-1 with PD-L1 and/or PD-L2. In some embodiments, a PD-1 checkpoint inhibitor reduces the negative co-stimulatory signal mediated by or through cell surface proteins expressed on T lymphocytes so as render a dysfunctional T-cell less dysfunctional (e.g., enhancing effector responses to antigen recognition). In some embodiments, the PD-1 checkpoint inhibitor is an anti-PD-1 antibody.

Thus, according to certain aspects of the present invention, the immune checkpoint therapy may comprise a monoclonal antibody against CTLA-4, PD-1, or PD-L1.

For example, the immune checkpoint inhibitor may be an inhibitor of PD-1. The immune checkpoint inhibitor may be an anti-PD-1 antibody, such as nivolumab. For example, the inhibitors of PD-1 biological activity (or its ligands) disclosed in U.S. Pat. No. 7,029,674. Exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

According to certain aspects, the immune checkpoint inhibitor is an inhibitor of PD-L1. Exemplary immune checkpoint inhibitors include antibodies (e.g., an anti-PD-L1 antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. An exemplary anti-PD-L1 antibody includes clone EH12. Exemplary antibodies against PD-L1 include: Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736 (Durvalumab).

According to certain aspects, the immune checkpoint inhibitor is an inhibitor of PD-L2 or reduces the interaction between PD-1 and PD-L2. Exemplary immune checkpoint inhibitors include antibodies (e.g., an anti-PD-L2 antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

According to certain aspects, the immune checkpoint inhibitor is an inhibitor of CTLA-4, such as an anti-CTLA-4 antibody. According to one aspect, the immune checkpoint inhibitor may be ipilimumab. The anti-CTLA-4 antibody may block the binding of CTLA-4 to CD80 (B7-1) and/or CD86 (B7-2) expressed on antigen presenting cells. Exemplary antibodies against CTLA-4 include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. According to certain aspects, the immune checkpoint inhibitor may be a nucleic acid inhibitor of CTLA-4 expression.

According to certain aspects, the immune checkpoint therapies include inhibitors of the lymphocyte activation gene-3 (LAG-3), such as IMP321, a soluble Ig fusion protein that activates dendritic cells; inhibitors of B7, such as antibodies against B7-H3 (e.g., MGA271) and B7-H4; and inhibitors against TIM3 (i.e., T-cell immunoglobulin domain and mucin domain 3).

Any suitable immune checkpoint inhibitor is contemplated for use with the compositions, dosage forms, and methods disclosed herein. The selection of the immune checkpoint inhibitor depends on multiple factors. For example, factors to be considered include any additional drug interactions of the immune checkpoint inhibitor, and the length for which the immune checkpoint inhibitor may be taken. In certain instances, the immune checkpoint inhibitor is an immune checkpoint inhibitor which may be taken long-term, for example chronically. Immune checkpoint therapies of the present invention may include immunostimulatory agents, T cell growth factors, an interleukin such as IL-7 or IL-15, an antibody, a vaccine such as a dendritic cell (DC) vaccine, or any combination thereof.

According to certain aspects of the present invention, the immune checkpoint therapy may include more than one modulator of an immune checkpoint protein. As such, the immune checkpoint therapy may comprise a first antibody or inhibitor against a first immune checkpoint protein and a second antibody or inhibitor against a second immune checkpoint protein. For example, according to certain aspects of the present invention, a first inhibitor may be an antibody against PD-1 and the second inhibitor may be an antibody against CTLA-4, or PD-L1, or PD-L2.

Proliferative Disorders

The compositions and methods of the present invention may be used to treat a proliferative disease or disorder. According to certain aspects of the present invention, the proliferative disease or disorder may be a cancer including, but not limited to, a hematologic malignancy, a solid tumor, a primary or a metastasizing tumor.

For example, the proliferative disorder may be one or more hematological cancers. Exemplary hematological cancers include at least B-cell acute lymphoid leukemia, T-cell acute lymphoid leukemia, acute lymphoid leukemia, chronic myelogenous leukemia, chronic lymphocytic leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell- or large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, asymptomatic myeloma, smoldering multiple myeloma, indolent myeloma, monoclonal gammapathy of undetermined significance, plasma cell dyscrasia, solitary myeloma, solitary plasmacytoma, extramedullary plasmacytoma, multiple plasmacytoma, systemic amyloid light chain amyloidosis, POEMS syndrome, and a combination thereof.

The proliferative disorder may be one or more solid cancers. Exemplary solid cancers include at least bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, primary mediastinal large B-cell lymphoma, diffuse large B-cell lymphoma, follicular lymphoma, transformed follicular lymphoma, splenic marginal zone lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B-cell malignancies, and any combination thereof.

According to certain aspects of the present invention, the hematological cancer or malignancy may be multiple myeloma, acute myeloid leukemia, myelodysplastic syndrome, and myeloproliferative neoplasm.

Methods of the Invention

The present invention includes methods for treating, ameliorating or reducing the severity of at least one symptom or indication, or inhibiting the growth of a cancer in a subject by administering a therapeutically effective amount of a radioimmunotherapy and a therapeutically effective amount of an immune checkpoint therapy. The present invention includes methods for initiating, enhancing, or prolonging an anti-tumor response in a subject by administering a therapeutically effective amount of a radioimmunotherapy and a therapeutically effective amount of an immune checkpoint therapy. The present invention includes methods for treating a proliferative disease or disorder in a subject by administering a therapeutically effective amount of a radioimmunotherapy and a therapeutically effective amount of an immune checkpoint therapy.

According to certain aspects of the present invention, the methods may treat patients with tumors having a standard or even high mutational burden, such as melanomas, renal cell carcinomas, and lung cancers, wherein the patients are poor responders or non-responders to standard immunotherapies (e.g., patients with T-cell exhaustion).

According to certain aspects, the methods may treat patients with tumors that are known to be immunologically cold. That is, the radioimmunotherapy administered in the methods may target antigens from tumors having a low mutational burden, such as antigens expressed by tumors of the pancreas, neuroblastomas, and hematological diseases.

According to certain aspects, the methods may treat patients with tumors or disorders that are hematopoietic in origin. That is, the radioimmunotherapy administered in the methods may target antigens from a hematological cell or a tumor cell having a hematopoietic origin.

According to certain aspects, the radioimmunotherapy and the immune checkpoint therapy may be administered simultaneously. As such, they may be provided as a single composition, or they may be provided as separate compositions administered simultaneously. The combination may be administered in a single dose. Alternatively, the combination may be administered according to a dosing schedule selected from the group consisting of once every 1, 2, 3, 4, 5, 6, 7, 8, 9, 10, 12, 14, 20, 24, or 28 days throughout a treatment period, wherein the treatment period includes at least two doses.

According to certain aspects, the radioimmunotherapy and the immune checkpoint therapy may be administered sequentially. Moreover, each therapy regime, i.e., radioimmunotherapy and immune checkpoint therapy, may be administered according to a specific dosing schedule, wherein the method provides for administration of each therapy according to the dosing schedule sequentially, i.e., the radioimmunotherapy dosing schedule is completed before the immune checkpoint therapy dosing schedule is started, or vice versa.

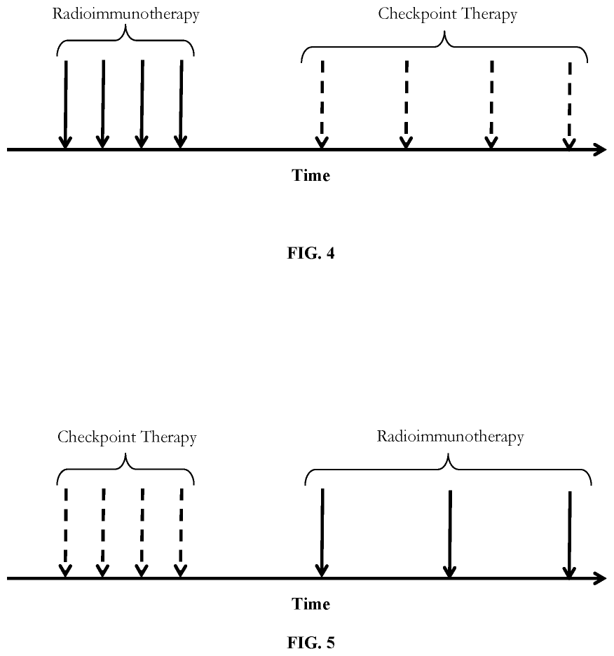

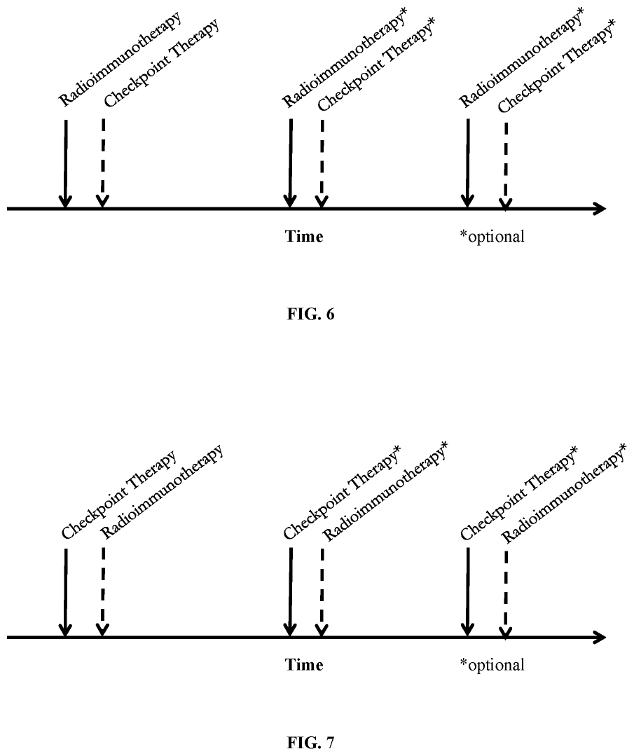

For example, the radioimmunotherapy may be administered in one or more doses prior to administration of the immune checkpoint therapy, as shown in FIG. 4, or the immune checkpoint therapy may be administered in one or more doses prior to administration of the radioimmunotherapy, as shown in FIG. 5.

According to certain aspects, more than one radioimmunotherapy may be administered to a patient, wherein a first and second radioimmunotherapy may be administered simultaneously, or sequentially. The immune checkpoint therapy may be administered before or after the first and second radioimmunotherapy or may be administered after the first radioimmunotherapy and before the second radioimmunotherapy.

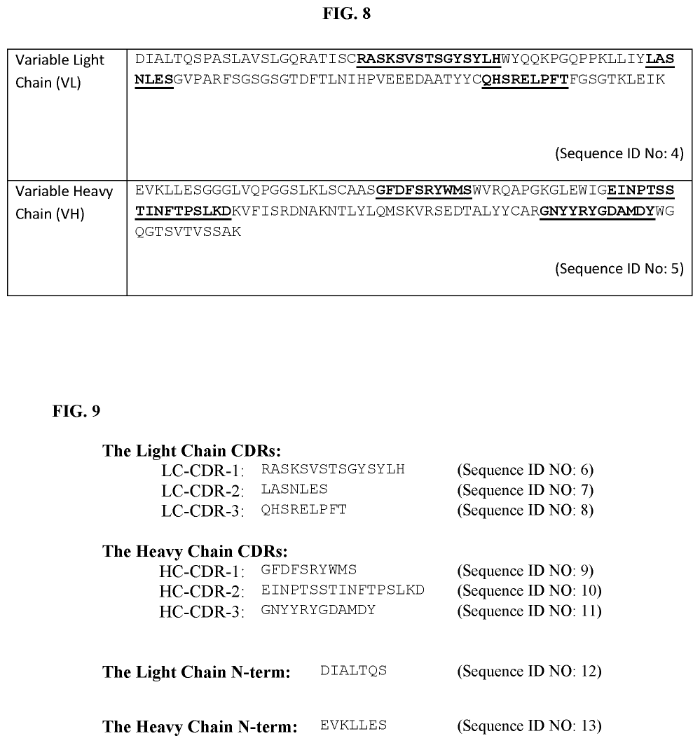

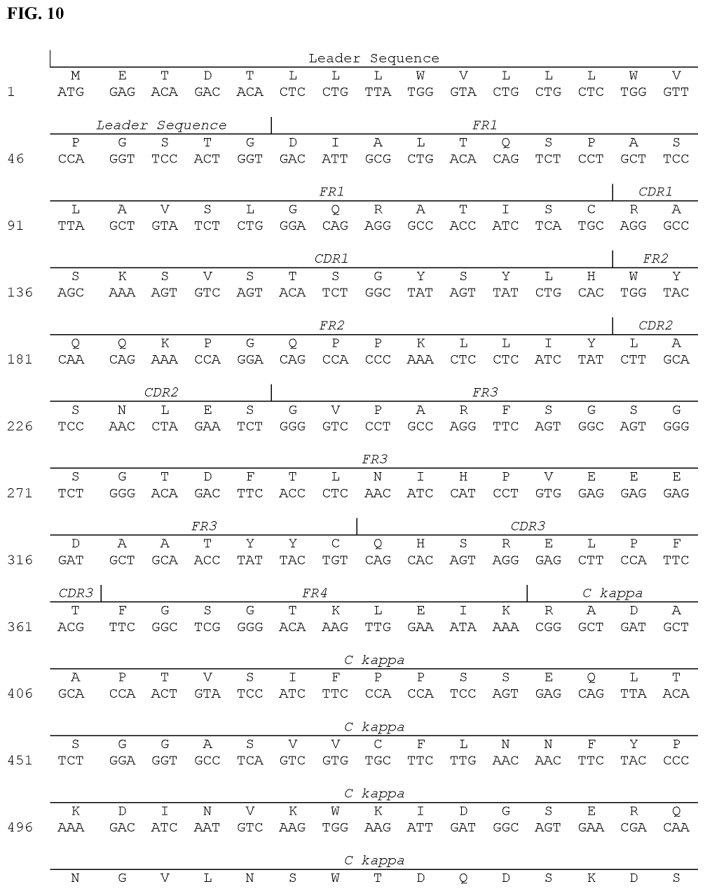

According to certain aspects of the present invention, the radioimmunotherapy and the immune checkpoint therapy may be administered according to specific dosing schedules that are carried out simultaneously. That is, doses of the radioimmunotherapy may be administered during the administration schedule of the immune checkpoint therapy, or vice versa. For example, doses of the radioimmunotherapy and the checkpoint immune therapy may be given as shown in FIG. 6 and FIG. 7, wherein individual doses of each therapeutic agent are administered in overlapping dosing schedules.

According to certain aspects of the methods of the present invention, the radioimmunotherapy may be administered in a single dose. Alternatively, the radioimmunotherapy may be administered according to a dosing schedule selected from the group consisting of once every 1, 2, 3, 4, 5, 6, 7, 8, 9, 10, 12, 14, 20, 24, or 28 days throughout a treatment period, wherein the treatment period includes at least two doses. The radioimmunotherapy may be administered during a weekly schedule, such as once every weekday but not on weekend days (Saturday or Sunday). Moreover, each dose may be the same, or may be different. For example, a first dose or set of doses of the radioimmunotherapeutic agent may be larger (induction dose(s)) than additional doses or sets of doses (continuation doses).

According to certain aspects of the present invention, the immune checkpoint therapy may be administered in a single dose. Alternatively, the immune checkpoint therapy may be administered according to a dosing schedule selected from the group consisting of once every 1, 2, 3, 4, 5, 6, 7, 8, 9, 10, 12, 14, 20, 24, or 28 days throughout a treatment period, wherein the treatment period includes at least two doses. The immune checkpoint therapy may be administered during a weekly schedule, such as once every weekday but not on weekend days (Saturday or Sunday). Moreover, each dose may be the same, or may be different. For example, a first dose or set of doses of the immune checkpoint therapy agent may be larger (induction dose(s)) than additional doses or sets of doses (continuation doses).

According to certain aspects of the present invention, the therapeutically effective amount of the radioimmunotherapy may comprise a radiation dose dependent on the selected radionuclide used for the labeling. For example, when a radionuclide such as ²²⁵Ac is selected for the radioimmunotherapy, the radiation dose may be about 0.1 to 20 uCi/kg patient body weight, such as 0.2 to 10 uCi/kg patient body weight, or 0.2 to 5 uCi/kg patient body weight, or 0.4 to 4 uCi/kg patient body weight, or 0.4 to 3 uCi/kg patient body weight, or even 0.4 to 2 uCi/kg patient body weight. Alternatively, when the radionuclide such as ¹³¹I is selected, the radiation dose may be up to 1000-fold higher. Preferred radiation doses for select radionuclides are described hereinabove in the definitions section of this disclosure.

The effective dose of the radioimmunotherapy generally comprises a protein dose of less than 16 mg/kg patient body weight, such as less than 10 mg/kg patient body weight, or less than 6 mg/kg patient body weight, or less than 5 mg/kg patient body weight, or less than 4 mg/kg patient body weight, or less than 3 mg/kg patient body weight, or even less than 2 mg/kg patient body weight. According to certain aspects, the protein dose may be from 0.1 mg/kg to 16 mg/kg body weight of the subject, such as 0.1 mg/kg to 10 mg/kg, or 0.1 mg/kg to 6 mg/kg, or 0.1 mg/kg to 4 mg/kg, or 0.1 mg/kg to 2 mg/kg, or 0.5 mg/kg to 16 mg/kg. or 2 mg/kg to 16 mg/kg, or 4 mg/kg to 16 mg/kg, or 6 mg/kg to 16 mg/kg.

According to certain aspects of the present invention, the effective dose of the radioimmunotherapy may comprise a protein dose based on the patient's body surface area, such as a dose of less than 10 mg/m², or 8 mg/m², or 6 mg/m², or 5 mg/m², or 4 mg/m², or 3 mg/m², or even 2 mg/m².

According to certain aspects of the present invention, the effective amount of the radioimmunotherapy may be a maximum tolerated dose (MTD) of the radioimmunotherapy, based on either or both of the protein dose and the radiation dose.

According to certain aspects of the present invention, the radioimmunotherapy may comprise a mixture of a radiolabeled fraction of an antibody and an un-labeled (e.g., “naked”) fraction of the antibody. The un-labeled fraction may comprise the same antibody against the same epitope as the labeled fraction. In this way, the total radioactivity of the radioimmunotherapy may be reduced or set while the overall antibody concentration may be varied. For example, the total protein concentration and the total radioactivity of the radioimmunotherapy may be independently varied based on the exact nature of the disease to be treated, age and weight of the patient, identity of the antibody, and the label (e.g., radionuclide) selected for labeling of the monoclonal antibody.

Exemplary doses for certain of these immune checkpoint therapies include individual doses of from 0.1 mg/kg to 50 mg/kg of the patient's body weight, such as 0.1-40 mg/kg, or 0.1-30 mg/kg, or 0.1-20 mg/kg, or 0.1-10 mg/kg, or 0.1-5 mg/kg, or 0.1-4 mg/kg, or 0.1-3 mg/kg, or 0.1-2 mg/kg, or 1-50 mg/kg, or 2-40 mg/kg, or 5-30 mg/kg, or 5-20 mg/kg, or 10-20 mg/kg, or 1-5 mg/kg, or 1-10 mg/kg. For example, dosing for pembrolizumab (anti-PD-1; Keytruda®) and nivolumab (anti-PD-1; Opdivo®) may be 1-5 mg/kg, such as 2 mg/kg or 3 mg/kg of the patient's body weight; and dosing for Durvalumab (anti-PD-L1; MEDI4736) may be 10 mg/kg to 20 mg/kg of the patient's body weight. Dosing for anti-CTLA-4 (Yervoy®) 1-15 mg/kg, such as 2 mg/kg or 3 mg/kg of the patient's body weight every three weeks for a maximum of 4 doses.

Additional Agents

The methods of the present invention, which include administration of a radioimmunotherapy and an immune checkpoint therapy, may further comprise administering one or more additional therapeutic agents. The additional therapeutic agents may be relevant for the disease or condition to be treated. Such administration may be simultaneous, separate or sequential with the administration of the effective amount of the radioimmunotherapy and the immune checkpoint therapy regimes detailed herein. For simultaneous administration, the agents may be administered as one composition, or as separate compositions, as appropriate.

Exemplary additional therapeutic agents include at least chemotherapeutic agents, anti-inflammatory agents, immunosuppressive agents, immunomodulatory agents, or a combination thereof. Exemplary chemotherapeutic and anti-inflammatory agents are well known in the art and within the scope of the presently disclosed invention.

According to certain aspects of the present invention, the one or more therapeutic agents may comprise an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

The therapeutic agents may be administered according to any standard dose regime known in the field. For example, therapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epothilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

Aspects of the Invention

The following aspects are disclosed in this application:

Aspect 1: A method for treating a subject having a proliferative disorder, the method comprising: administering to the subject a therapeutically effective amount of an immune checkpoint therapy; and after at least one week, administering to the subject a therapeutically effective amount of a radioimmunotherapy.

Aspect 2: A method for treating a subject having a proliferative disorder, the method comprising: administering to the subject a therapeutically effective amount of an radioimmunotherapy; and after at least one week, administering to the subject a therapeutically effective amount of an immune checkpoint therapy.

Aspect 3: A method for treating a subject having a proliferative disorder, the method comprising: administering to the subject a therapeutically effective amount of an radioimmunotherapy; and administering to the subject a therapeutically effective amount of an immune checkpoint therapy.

Aspect 4: The method according to any one of aspects 1 to 3, wherein administration of the radioimmunotherapy and/or immune checkpoint therapy is according to a dosing schedule selected from the group consisting of once every 1 day, 2 days, 3 days, 4 days, 5 days, 6 days, 7 days, 10 days, 12 days, 14 days, 21 days, or 28 days, wherein the treatment period includes at least two doses.

Aspect 5: The method according to any one of aspects 1 to 4, wherein the radioimmunotherapy comprises a radionuclide selected from the group consisting of ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

Aspect 6: The method according to any one of aspects 1 to 5, wherein the radioimmunotherapy comprises a radionuclide selected from the group consisting of ¹³¹I, ¹⁷⁷Lu, or ²²⁵Ac.

Aspect 7: The method according to any one of aspects 1 to 6, wherein the radioimmunotherapy comprises a radionuclide complexed by a chelating agent attached to the antibody of the radioimmunotherapy, where the chelating agent comprises 1,4,7,10-tetraazacyclododecane-1,4,7,10-tetraacetic acid (DOTA) or a derivative thereof.

Aspect 8: The method according to any one of aspects 1 to 7, wherein the radioimmunotherapy comprises an antibody against CD19, CD20, CD22, CD30, CD33, CD38, CD45, HER3, DR5, CD123, CD138, CS-1, B-cell maturation antigen (BCMA), MAGEA3, MAGEA3/A6, KRAS, CLL1, MUC-1, HER2, IL13Ra2, and EphA2, EpCam, GD2, GPA7, PSCA, EGFR, EGFRvIII, ROR1, GPC3, CEA, Mesothelin, PSMA, or a combination thereof.

Aspect 9: The method according to any one of aspects 1 to 7, wherein the radioimmunotherapy comprises an antibody against a protein product of a gene mutated in acute myeloid leukemia, wherein the gene is NPM1, Flt3, TP53, CEBPA, KIT, N-RAS, MLL, WT1, IDH1/2, TET2, DNMT3A, ASXL1, or a combination thereof.

Aspect 10: The method according to any one of aspects 1 to 7, wherein the radioimmunotherapy comprises an antibody against CD33, CD38, CD45, HER3, DR5, or a combination thereof.

Aspect 11: The method according to aspect 10, wherein the anti-CD33 antibody comprises lintuzumab; or the anti-CD38 comprises daratumumab; or the anti-CD45 antibody comprises BC8.

Aspect 12: The method according to any one of aspects 1 to 11, wherein the radioimmunotherapy comprises a first radioimmunotherapy against CD33, CD38, CD45, HER3 or DR5, and a second radioimmunotherapy against a different one of CD33, CD38, CD45, HER3 or DR5.

Aspect 13: The method according to aspect 12, wherein the first radioimmunotherapy is an antibody against CD33, CD38, or CD45, and the second radioimmunotherapy is an antibody against HER3 or DR5.

Aspect 14: The method according to aspects 12 or 13, wherein the first and second radioimmunotherapies are delivered simultaneously or sequentially.

Aspect 15: The method according to aspects 12 or 13, wherein the first radioimmunotherapy is administered before the immune checkpoint therapy and the second radioimmunotherapy is administered after the immune checkpoint therapy; or wherein the second radioimmunotherapy is administered before the immune checkpoint therapy and the first radioimmunotherapy is administered after the immune checkpoint therapy.

Aspect 16: The method according to any one of aspects 1 to 15, wherein the therapeutically effective amount of the radioimmunotherapy comprises a protein dose of less than 16 mg/kg body weight of the subject; or less than 10 mg/kg body weight of the subject; or less than 6 mg/kg body weight of the subject; or from 0.1 mg/kg to 16 mg/kg body weight of the subject.

Aspect 17: The method according to any one of aspects 1 to 16, wherein the therapeutically effective amount of the radioimmunotherapy is a maximum tolerated dose (MTD).

Aspect 18: The method according to any one of aspects 1 to 16, wherein the therapeutically effective amount of the radioimmunotherapy comprises a label dose of 0.05 to 10 uCi/kg body weight of the subject; or 0.1 to 6 uCi/kg body weight of the subject; or 0.2 to 5 uCi/kg body weight of the subject.

Aspect 19: The method according to any one of aspects 1 to 16, wherein the therapeutically effective amount of the radioimmunotherapy comprises a label dose of 0.05 to 10 mCi/kg body weight of the subject; or 0.1 to 6 mCi/kg body weight of the subject; or 0.1 to 5 mCi/kg body weight of the subject; or 0.1 to 3 mCi/kg body weight of the subject.

Aspect 20: The method according to any one of aspects 1 to 16, wherein the therapeutically effective amount of the radioimmunotherapy comprises a single dose that delivers less than 2Gy, or less than 8 Gy, such as doses of 2 Gy to 8 Gy, to the subject.

Aspect 21: The method according to any one of aspects 1 to 20, wherein the immune checkpoint therapy comprises an antibody against CTLA-4, PD-1, TIM-3, VISTA, BTLA, LAG-3, TIGIT, CD28, OX40, GITR, CD137, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or a combination thereof.

Aspect 22: The method according to any one of aspects 1 to 20 wherein the immune checkpoint therapy comprises an antibody against PD-1, PD-L1, PD-L2, CTLA-4, or a combination thereof.

Aspect 23: The method according to any one of aspects 1 to 22, wherein the proliferative disorder is a hematological cancer selected from one or more of multiple myeloma, acute myeloid leukemia, myelodysplastic syndrome, and myeloproliferative neoplasm.

Aspect 24: The method according to any one of aspects 1 to 23, wherein the radioimmunotherapy comprises BC8, wherein the BC8 comprises a light chain having the amino acid sequence as set forth in SEQ ID NO:1, or a light chain N-terminal amino acid sequence as set forth in SEQ ID NO: 9.

Aspect 25: The method according to any one of aspects 1 to 24, wherein the radioimmunotherapy comprises BC8, wherein the light chain of the BC8 comprises at least one complementarity determining region having the amino acid sequence as set forth in SEQ ID NO:3, SEQ ID NO:4, or SEQ ID NO:5.

Aspect 26: The method according to any one of aspects 1 to 23, wherein the radioimmunotherapy comprises BC8, wherein the BC8 comprises a light chain having the amino acid sequence set forth in SEQ ID NO:12 or SEQ ID NO:13.

Aspect 27: The method according to any one of aspects 1 to 26, wherein the radioimmunotherapy comprises BC8, wherein the BC8 comprises a heavy chain having the amino acid sequence set forth in SEQ ID NO:2, or a heaving chain N-terminal amino acid sequence as set forth in SEQ ID NO: 10.

Aspect 28: The method according to any one of aspects 1 to 27, wherein the radioimmunotherapy comprises BC8, wherein the heavy chain of the BC8 comprises at least one complementarity determining region having the amino acid sequence as set forth in SEQ ID NO:6, SEQ ID NO:7, or SEQ ID NO:8.

Aspect 29: The method according to any one of aspects 1 to 27, wherein the radioimmunotherapy comprises BC8, wherein the BC8 comprises a heavy chain having the amino acid sequence set forth in SEQ ID NO:15 or SEQ ID NO:16.

Aspect 30: The method according to any one of aspects 1 to 28, wherein the radioimmunotherapy comprises BC8, and the heavy chain of the BC8 comprises the amino acid ASP or ASN at position 141 (relative to the N-terminal amino acid).

### EXAMPLES

### Example 1: Toxicity of Actinium-225 Labeled CD33

According to certain aspects of the present invention, the radioimmunotherapy may comprise an Actinium-225 (Ac²²⁵) labeled monoclonal antibody against CD33. Lintuzumab conjugated with Actinium-225 (Ac²²⁵) was tested for cytotoxicity against specific cell types that express CD33. For example, suspensions of HL60 (leukemia cells) were incubated with various doses of radiolabeled lintuzumab (lintuzumab-Ac²²⁵), and the dose at which 50% of the cells were killed (LD₅₀) was found to be 8 pCi per mL of cell suspension.

In studies to access the reactivity of the radiolabeled lintuzumab with peripheral blood and bone marrow cells from nonhuman primate and human frozen tissues, the radiolabeled lintuzumab showed reactivity with mononuclear cells only, demonstrating specificity. Moreover, in studies to determine the stability of the radiolabel on the antibody, 10 normal mice (8 week old Balb/c female mice from Taconic, Germantown, N.Y.) were injected in the tail with 300 nCi radiolabeled lintuzumab (in 0.12m1). Serum samples taken over a 5 day period showed that the Actinium-225 remained bound to the lintuzumab, demonstrating the stability of the radiolabel on the antibody in vivo.

A maximum tolerated dose (MTD) of a single injection of the radiolabeled lintuzumab was determined to be 3uCi/kg patient weight. As a split dose (e.g., 2 equal doses administered 4-7 days apart), the MTD was determined to be 2uCi/kg per dose, or 4uCi/kg total. This data was determined by injections into patients with relapsed/refractory AML: 21 patients were injected with increasing doses of the radiolabeled lintuzumab—0.5uCi/kg to 4uCi/kg. Determination of MTD was based on the severity of the adverse effects observed at each dose level. Anti-leukemic effects included elimination of peripheral blood blasts in 13 of 19 evaluable patients. Twelve of 18 patients who were evaluable at 4 weeks following treatment had reductions in bone marrow blasts, including nine with reductions ≥50%. Three patients treated with 1 uCi/kg, 3uCi/kg and 4uCi/kg respectively had ≤5% blasts after therapy.

### Example 2: CD33 Human Maximal Tolerated Dose and Efficacy

A Phase I trial will be used to determine the MTD of fractionated doses of lintuzumab-Ac²²⁵ followed by Granulocyte Colony Stimulating factor (GCSF) support in each cycle. A cycle in general is approximately 42 days. A cycle starts with administration of a fractionated dose of Lintuzumab-Ac²²⁵ on Day 1 followed by the administration of GCSF on Day 9 and continuing GCSF per appropriate dosing instructions until absolute neutrophil count (ANC) is greater than 1,000, which is expected to occur within 5-10 days. On Days 14, 21, 28, 35 and 42 peripheral blood will be assessed for paraprotein burden. A bone marrow aspirate will be performed to assess plasmocyte infiltration on Day 42. If a response is a partial response or better but less than a complete response on Day 42, and the patient remains otherwise eligible, the patient will be re-dosed in a new cycle at the same dose level no sooner than 60 days after Day 1 of the first cycle. In absence of dose limiting toxicities, cycles will continue using the above described algorithm until the patient has received a cumulative dose of 4 μCi/kg of lintuzumab-Ac²²⁵.

### Example 3: CD33 Combination Therapy with a Chemotherapeutic Agent

In a phase 1 clinical trial, eighteen patients with relapsed AML were treated with ²²⁵Ac-lintuzumab administered in fractionated doses on days 1 and 8 combined with low dose Ara-C (LDAC). Treatment was found to induce remissions in older patients with untreated AML at doses above 0.5 uCi/kg/fraction. In a Phase 2 portion of the clinical trial, thirteen patients with initial presentation of AML who were considered to be unfit for cytotoxic chemotherapy were treated with ²²⁵Ac-lintuzumab administered in fractionated doses on days 1 and 8 without LDAC. Preliminary data are available on 9 with median age 75 years (range, 65-82) and PS median 2 (0-1 in 2 patients, 2 in 3 patients, & 3 in 2 patients). Six (67%) had prior treatment for AHDs (5 MDS, 1 atypical CML). At baseline, 5 patients had ANC≥500/μL, only 2 had ANC≥1000/μL, and only 1 had platelets >50,000/μL.

Myelosuppression was seen in all evaluable patients including grade 4 thrombocytopenia with marrow aplasia for >6 weeks following therapy in 3 patients. The only Grade >3 non-hematologic toxicities reported in ≥1 pt were pneumonia and cellulitis. Veno-occlusive disease did not occur. The 30-day mortality rate was 33% (disease progression, acute on chronic respiratory failure, and post-traumatic intracranial hemorrhage after a fall).

Objective responses were documented in 5 of the 9 patients (56%): 2 Complete Remissions with incomplete platelet count recovery (CRp) and 3 Complete Remissions with incomplete hematologic recovery (CRi). Two patients had resistant disease.

Median time to neutrophil recovery (ANC≥500/μL) was 36 days (range 20-60) from the first dose of ²²⁵Ac-lintuzumab. The two patients with CRp had neutrophil recovery at Days 60 and 36. Two of the patients with CRi had not reached ANC≥500/μL when they expired from infection on days 65 and 56, and the third is at day 66+ without ANC recovery. Since patients with antecedent hematologic disorders (AHDs) may not have capacity to recover to normal neutrophil production, patients without AHDs may be more informative. Of the 3 patients without AHDs, 1 had ANC recovery at Day 36, 1 had death from infection at Day 56 without ANC recovery, and 1 is pending ANC recovery at Day 66+. No patients reached platelet counts >20,000/μL without transfusions.

Preliminary data from this Phase 2 trial of ²²⁵Ac-lintuzumab monotherapy at 2 μCi/kg/fraction document a 56% response rate in older patients unfit for intensive therapy, many with AHDs. As myelosuppression at this dose was considered to be longer than acceptable in this population, accrual to this study will continue at 1.5 μCi/kg/fraction with the goal to shorten recovery times.

### Example 4: Production of the Anti-CD45 Immunoglobulin BC8

According to certain aspects of the present invention, the radioimmunotherapy may comprise a monoclonal antibody against CD45, such as BC8 or a chimeric version of BC8 (BC8c). The murine anti-CD45 mAb BC8 was prepared from a hybridoma (ATCC No. HB-10507) that was initially developed by fusing mouse myeloma NS1 cells with spleen cells from a BALB/C mouse hyperimmunized with human phytohemagglutinin (PHA)-stimulated mononuclear cells. The original fused cells, after screening for microbial contaminations, were cultured using the JRH-Biosciences EXCell 300 medium supplemented with 1-2% Fetal Bovine Serum (FBS).

The hybridoma cell line was adapted for culture in a serum-free culture medium. Briefly, the cells in culture were slowly and gradually weaned of the serum albumin using the combo medium supplemented with glutamine, cholesterol, insulin and transferrin. The cells were then grown in up to 500 L scale to a density of >1×10⁶ cells/ml. The medium was harvested and processed for the purification of the anti-CD45 antibody using a combination of cation exchange chromatography, protein-A chromatography, and anion exchange membrane separation. The purified antibody was concentrated by nano-filtration (30 kD cutoff). The concentration of the purified product was measured at 5.2 mg/ml and was stored at 2-8° C.

The purified antibody was characterized by SDS-PAGE, IEF and SEC-HPLC techniques. A single product peak (99.4%) was recorded with SEC-HPLC with about 0.6% aggregates. The non-reducing SDS-PAGE showed a single band for the antibody. The SDS-PAGE under reduced conditions confirmed the presence of the light and the heavy chains (99.9% together).

### Example 5: Sequencing of the Anti-CD45-Immunoglobulin BC8

Total RNA was isolated from the hybridoma cells following the technical manual of Trizol® Reagent. The total RNA was analyzed by agarose gel electrophoresis and was reverse transcribed into cDNA using isotype-specific anti-sense primers or universal primers following the technical manual of PrimeScript™ 1st Strand cDNA Synthesis Kit. The antibody fragments of VH, VL, CH and CL were amplified and were separately cloned into a standard cloning vector using standard molecular cloning procedures. Colony PCR screening was performed to identify clones with inserts of correct sizes. More than five single colonies with inserts of correct sizes were sequenced for each antibody fragment. The complete nucleotide sequence of the light and the heavy chains are shown in FIGS. 10 and 11.

The anti-CD45-immunoglobulin (i.e., BC8 antibody) was sequenced using the mass spectrometry peptide mapping approach. The BC8 antibody was de-glycosylated, reduced and digested with individual enzymes; trypsin, Lys-C and chymotrypsin. The peptide fragments were then analyzed by the LC-coupled mass spectrometry technique using the MS/MS fragmentation analysis approach. Protein sequencing of the heavy and light chains of the BC8 antibody showed that the actual amino acid sequence differs from that predicted by the DNA sequence by only a single amino acid in the heavy chain. As highlighted in FIG. 11, the codon that codes for the amino acid at position 141 predicts an ASN-141 and not the actual ASP-141 found by protein sequencing. Moreover, sequencing of various batches of the protein indicated differing amounts of the ASP and ASN at position 141.

1. A method for treating a subject having a proliferative disorder, the method comprising:
administering to the subject a therapeutically effective amount of an immune checkpoint therapy; and
administering to the subject a therapeutically effective amount of a radioimmunotherapy,
wherein the radioimmunotherapy comprises a radionuclide selected from the group comprising ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or a combination thereof.

2. The method of claim 1, wherein the radioimmunotherapy is administered at least one week before the immune checkpoint therapy; or wherein the immune checkpoint therapy is administered at least one week before the radioimmunotherapy.

3. (canceled)

4. The method of claim 1, wherein the radioimmunotherapy comprises an antibody against CD19, CD20, CD22, CD30, CD33, CD38, CD45, CD123, CD138, CS-1, B-cell maturation antigen (BCMA), MAGEA3, MAGEA3/A6, KRAS, CLL1, MUC-1, HER2, HER3, DR5, IL13Ra2, and EphA2, EpCam, GD2, GPA7, PSCA, EGFR, EGFRvIII, ROR1, GPC3, CEA, Mesothelin, PSMA, or a combination thereof.

5. The method of claim 1, wherein the radioimmunotherapy comprises an antibody against a protein product of a gene mutated in acute myeloid leukemia, wherein the gene is NPM1, Flt3, TP53, CEBPA, KIT, N-RAS, MLL, WT1, IDH1/2, TET2, DNMT3A, ASXL1, or a combination thereof.

6. The method of claim 1, wherein the radioimmunotherapy comprises an antibody against CD33, CD38, CD45, HER3, DR5, or a combination thereof.

7. The method of claim 1, wherein the radioimmunotherapy comprises an antibody against CD33, HER3, or DR5 labeled with a radionuclide selected from the group consisting of ¹³¹I, ¹⁷⁷Lu, and ²²⁵Ac.

8. The method of claim 7, wherein the radionuclide is ²²⁵Ac, and the therapeutically effective amount of the radioimmunotherapy comprises a radiation dose of 0.1 to 10 uCi/kg body weight of the subject; or 0.2 to 6 uCi/kg body weight of the subject; or 0.4 to 5 uCi/kg body weight of the subject.

9. The method of claim 7, wherein the radionuclide is ¹³¹I, and the therapeutically effective amount of the radioimmunotherapy comprises a radiation dose of 25 mCi to 500 mCi; or 50 mCi to 400 mCi.

10. The method of claim 1, wherein the radioimmunotherapy comprises a radionuclide label complexed by a chelating agent attached to an antibody, wherein the chelating agent comprises 1,4,7,10-tetraazacyclododecane-1,4,7,10-tetraacetic acid (DOTA) or a derivative thereof.

11. The method of claim 1, wherein the therapeutically effective amount of the radioimmunotherapy is a maximum tolerated dose (MTD).

12. The method of claim 1, wherein the immune checkpoint therapy comprises an antibody against CTLA-4, PD-1, TIM-3, VISTA, BTLA, LAG-3, TIGIT, CD28, OX40, GITR, CD137, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or a combination thereof.

13. The method of claim 1, wherein the immune checkpoint therapy comprises an antibody against PD-1, PD-L1, PD-L2, CTLA-4, or a combination thereof.

14. The method of claim 1, wherein the radioimmunotherapy comprises one of: (a) a first and second radioimmunotherapy, both of which are administered at least one week before the immune checkpoint therapy; (b) a first radioimmunotherapy administered at least one week before the immune checkpoint therapy and a second radioimmunotherapy administered at least one week after the immune checkpoint therapy; (c) a first radioimmunotherapy administered with the immune checkpoint therapy and a second radioimmunotherapy administered at least one week after the immune checkpoint therapy; or (d) a first and second radioimmunotherapy, both of which are administered at least one week after the immune checkpoint therapy.

15. The method of claim 14, wherein the first radioimmunotherapy comprises an antibody against one of CD33, CD38, CD45, DR5, or HER3, and the second radioimmunotherapy comprises an antibody against another of CD33, CD38, CD45, DR5, or HER3.

16. The method of claim 1, wherein the proliferative disorder is a hematological cancer selected from one or more of multiple myeloma, acute myeloid leukemia, myelodysplastic syndrome, and myeloproliferative neoplasm.

17. A method for treating a subject having a proliferative disorder, the method comprising:
administering to the subject a therapeutically effective amount of an immune checkpoint therapy selected from the group consisting of PD-1, PD-L1, PD-L2, CTLA-4, or a combination thereof; and
after at least one week, administering to the subject a therapeutically effective amount of a radioimmunotherapy selected from the group consisting of CD33, CD38, CD45, DR5, HER3, or a combination thereof, wherein the radioimmunotherapy is labeled with a radionuclide selected from the group consisting of ¹³¹I, ¹⁷⁷Lu, and ²²⁵Ac.

18. A method for treating a subject having a proliferative disorder, the method comprising:
administering to the subject a therapeutically effective amount of a radioimmunotherapy selected from the group consisting of CD33, CD38, CD45, DR5, HER3, or a combination thereof, wherein the radioimmunotherapy is labeled with a radionuclide selected from the group consisting of ¹³¹I, ¹⁷⁷Lu, and ²²⁵Ac; and
after at least one week, administering to the subject a therapeutically effective amount of an immune checkpoint therapy selected from the group consisting of PD-1, PD-L1, PD-L2, CTLA-4, or a combination thereof.

19. The method of claim 17, wherein the radionuclide is ²²⁵Ac, and the therapeutically effective amount of the radioimmunotherapy comprises a radiation dose of 0.1 to 10 uCi/kg body weight of the subject, and a protein dose of 0.1 mg/kg to 16 mg/kg body weight of the subject.

20. The method of claim 17, wherein the radionuclide is ¹³¹I, and the therapeutically effective amount of the radioimmunotherapy comprises a radiation dose of 0.1 to 12 mCi/kg body weight of the subject, and a protein dose of 0.1 mg/kg to 16 mg/kg body weight of the subject.

21. The method of claim 18, wherein the radionuclide is ²²⁵Ac, and the therapeutically effective amount of the radioimmunotherapy comprises a radiation dose of 0.1 to 10 uCi/kg body weight of the subject, and a protein dose of 0.1 mg/kg to 16 mg/kg body weight of the subject.
